SEQUENCE LISTING (4 sequences)

SEQ ID NO: 1 - DNA, 57 bp, Artificial Sequence
  Primer
ccggctgtagcccatgttgtagcaactcgagttgctacaacatgggctacagttttt

SEQ ID NO: 2 - DNA, 57 bp, Artificial Sequence
  Primer
ccggcgggattctttccaaacacttctcgagaagtgtttggaaagaatcccgttttt

SEQ ID NO: 3 - DNA, 57 bp, Artificial Sequence
  Primer
ccgggcacgacaaatacgttgaaatctcgagatttcaacgtatttgtcgtgcttttt

SEQ ID NO: 4 - DNA, 57 bp, Artificial Sequence
  Primer
ccggcaacaagatgaagagcaccaactcgagttggtgctcttcatcttgttgttttt

(19) United States
(12) Patent Application Publication
(10) Pub. No.: US 2015/0258194 A1
(43) Pub. Date: Sep. 17, 2015

(54) MODULATING TRANSENDOTHELIAL MIGRATION AND RECRUITMENT OF GRANULOCYTES BY MODULATING C-MET PATHWAY

(71) Applicants: VIB VZW, Gent (BE); Life Sciences Research Partners VZW, Leuven (BE); Katholieke Universiteit Leuven, K.U.Leuven R&D, Leuven (BE)

(72) Inventors: Massimiliano Mazzone, Kessel-Lo (BE); Veronica Finisguerra, Leuven (BE)

(21) Appl. No.: 14/424,033

(22) PCT Filed: Sep. 2, 2013
(86) PCT No.: PCT/EP2013/068101

§ 371 Date: Feb. 25, 2015

Related U.S. Application Data
(60) Provisional application No. 61/695,952, filed on Aug. 31, 2012.

(51) Int. Cl. A61K 39/395; A61K 45/06; A61K 38/19; C07K 16/30

(52) U.S. Cl. CPC A61K 39/39558; C07K 16/3076; A61K 45/06; A61K 38/191; C07K 2317/76; C07K 2317/24

(57) ABSTRACT

Disclosed are granulocytes and their role in both cancer and inflammation. More particularly, it was found that c-Met expressed by granulocytes is important in transmigration and recruitment of the granulocytes. It is shown that reducing c-Met-mediated transmigration of granulocytes sustains tumor progression, indicating that c-Met-mediated granulocyte transmigration should actually be maintained because it is beneficial in treatment of cancers, particularly cancers that otherwise show resistance to c-Met inhibition. Reducing c-Met-mediated transmigration on the other hand is particularly useful in conditions characterized by an excessive immune response, such as asthma.

### CROSS-REFERENCE TO RELATED APPLICATIONS

This application is a national phase entry under 35 U.S.C. §371 of International Patent Application PCT/EP2013/068101, filed Sep. 2, 2013, designating the United States of America and published in English as International Patent Publication WO 2014/033298 A2 on Mar. 6, 2014, which claims the benefit under Article 8 of the Patent Cooperation Treaty and under 35 U.S.C. §119(e) to U.S. Provisional Patent Application Ser. No. 61/695,952, filed Aug. 31, 2012.

### TECHNICAL FIELD

The application relates to medicine, biotechnology, and granulocytes and their role in both cancer and inflammation. More particularly, it was found that c-Met expressed by granulocytes is important in transmigration and recruitment of the granulocytes, particularly neutrophils. Increasing c-Met-mediated transmigration of granulocytes is beneficial in treatment of cancers, particularly cancers that otherwise show resistance to c-Met inhibition. Reducing c-Met-mediated transmigration on the other hand is particularly useful in conditions characterized by an excessive immune response, particularly a granulocyte- or neutrophil-mediated immune response, such as asthma.

### BACKGROUND

MET is the tyrosine kinase receptor for Hepatocyte Growth Factor (HGF) and its activation contributes to a plethora of biological processes including proliferation, survival, motility, and differentiation of epithelial, endothelial, neuronal, and hematopoietic cells^{1,2}. During embryogenesis, MET or HGF is required for placenta and liver development, and also for the directional migration of myoblasts from the somites to the limbs ^{1,2}. In adults, the expression of both MET and HGF is low but the reactivation of this pathway is necessary during tissue damage when cells have to reacquire their ability to proliferate and migrate in order to allow organ repair or regeneration ¹.

MET is re-expressed in many human tumors as well 3. In this context, the transcriptional upregulation of MET is induced by the alteration of other genes 4-6 or by microenvironmental stimuli such as hypoxia or tumor cytokines that include interleukin (IL)-1, IL-6 and tumor necrosis factor-α (TNF-α) ^{7,8}. In a fraction of cases, MET is constitutively activated because of genomic amplification or point mutations of the MET proto-oncogene, or by the presence of ligand autocrine loops ^{3,9,10}. High levels of MET and/or HGF correlate with the aggressive phenotype of different carcinomas, including those of the prostate, stomach, pancreas, thyroid, lung and breast ^{3,11}.

MET activation has been involved in all the steps that allow cancer cells to grow and disseminate distantly, thus forming metastasis ^{1,11} For this reason, a lot of effort has been invested to demonstrate the efficacy of MET inhibition in pre-clinical models ¹²⁻¹⁷. To date, about twenty drugs blocking MET (or HGF) are being explored in Phase I, Phase II, and Phase III clinical trials across multiple tumor types ^{3,13}. Preliminary data demonstrate promising clinical activity of these agents especially on MET-driven tumors, along with an acceptable toxicity profile ^{3,14}. The effect of MET inhibitors on tumors that do not display aberrant MET hyperactivation and on MET-expressing cancer-associated stromal cells is less clear.

Notwithstanding the progress made, drug resistance continues to be the single most important cause of cancer treatment failure, and understanding the mechanisms of drug resistance remains a major hurdle in treating patients with recurrent disease.

Despite its expression in several cancer-associated cells, little if any is known about the functional role of c-Met in stromal cells during cancer progression. Cancer cells are not isolated, but rather subsist in a rich microenvironment provided by fibroblasts, endothelial cells (ECs), pericytes, adipocytes, and immune cells. MET expression has been reported in several of these cell types, including ECs, pericytes, monocytes, macrophages, dendritic cells, and lymphocytes ¹⁸⁻²⁵. However, little is known about the expression and biological role of MET in these stromal cells during cancer progression. Tumor response to anti-MET therapies has earlier been evaluated by analyzing human tumor xenografts in immunodeficient mice that partly or completely lack an immune system and, thus, also the immune modulatory activity on other cells, which influences the overall behavior of neoplastic and stromal cells ^{12,14-17}. We, therefore, evaluated if and how the inhibition of c-Met in the stroma influences tumor progression, disclosing possible modes of resistance to c-Met inhibitors in tumor treatment and, thus, opening novel perspectives for the improvement of existing anti-cancer therapies.

### SUMMARY

To study the role of c-Met on the tumor stroma, c-Met was selectively inhibited in the hematopoietic and endothelial cell lineage (see examples section). Surprisingly, while c-Met has a dispensable role in the endothelium, its deletion in the hematopoietic lineage fostered the tumor growth resulting in a larger tumor with increased metastasis. Further analysis revealed that this is due to decreased recruitment and infiltration of granulocytes, particularly neutrophils, indeed, inhibition of c-Met does not change infiltration of other inflammatory cells.

The link between c-Met and granulocytes was unknown and completely unexpected, but it has important consequences. The Met pathway is one of the most frequently dysregulated pathways in cancer, and c-Met inhibition is generally considered a promising therapeutic strategy for many forms of cancer. However, here it is shown that c-Met should not be inhibited in granulocytes, as this interferes with their recruitment and diapedesis, effectively resulting in a pro-tumoral response. Thus, c-Met activity (or the c-Met induced transmigration pathway) should be maintained in granulocytes even when it is inhibited in tumors.

Moreover, in other diseases, such as asthma, granulocyte (and in particular eosinophil and/or neutrophil) infiltration lies at the heart of the disease (Monteseirin, J Investig Allergol Clin Immunol. 19(5):340-54, 2009). The excessive recruitment and infiltration of granulocytes (and resulting tissue damage) is also seen in other disease states such as adult respiratory distress syndrome (Craddock et al., New Engl J Med 296:769-774, 1977), ischemia/reperfusion (I/R)-mediated renal, cardiac and skeletal muscle injury, rheumatoid arthritis (Weissmann and Korchak, Inflammation 8 Suppl:S3-14, 1984) and inflammatory bowel diseases such as Crohn's disease and ulcerative colitis (Wandall, Scand J Gastroenterol 20:1151-1156, 1985). Preventing granulocyte infiltration in these diseases would be a major step forward, and c-Met inhibition allows specific targeting of granulocytes while not affecting infiltration of other inflammatory cell types.

Provided are methods of modulating transendothelial migration and/or recruitment of granulocytes, the methods comprising modulating the c-Met pathway in the granulocytes. Most particularly, the granulocytes are neutrophils. According to these embodiments, methods of modulating transendothelial migration and/or recruitment of neutrophils are provided, comprising modulating the c-Met pathway in the neutrophils.

According to a first aspect, granulocyte transmigration and/or recruitment is enhanced by enhancing the c-Met pathway. According to particular embodiments, enhancing the c-Met pathway can be done by increasing β2-integrin expression and/or activation. According to further particular embodiments, increasing β2-integrin activation can be done by using an antibody.

According to specific embodiments, increasing β2-integrin activation (in the granulocytes) is done in presence of a c-Met inhibitor (particularly one that is not restricted to the granulocytes, but is used systemically or topically in another tissue or cell type than the granulocytes). According to particular embodiments, the c-Met inhibitor is an antibody. According to further particular embodiments, the c-Met inhibitor is onartuzumab, i.e., the MetMAb antibody.

According to particular embodiments, the granulocytes wherein the c-Met pathway is modulated are (at least in part) neutrophils.

The methods where concomitant β2-integrin activation and c-Met inhibition is envisaged are particularly suited for the treatment of cancer, most particularly cancer that is resistant or refractory against c-Met inhibitors alone. Accordingly, methods are provided to treat a subject with cancer, comprising administering a c-Met pathway enhancer (such as a β2-integrin activator) and a c-Met inhibitor to the subject in need thereof. Most particularly, the c-Met pathway enhancer is effective in the granulocytes of the subject, while the c-Met inhibitor is effective in the tumor of the patient.

According to a further aspect, granulocyte transmigration and/or recruitment is decreased by inhibiting the c-Met pathway. Most particularly, the c-Met pathway is inhibited by inhibiting c-Met. It is particularly envisaged that inhibition of c-Met is done with an antibody, such as, e.g., the onartuzumab (MetMAb) antibody.

As neutrophil-associated pro-tumorigenic effects are mainly dependent on TGF-β signaling and inhibition of TGF-β enables the N2, anti-tumoral, phenotype of neutrophils³³, the combined administration of a c-Met inhibitor and a TGF-β inhibitor to a subject in need, thereof, is also envisaged herein. Likewise, combinations of c-Met inhibitors and TGF-β inhibitors are provided. They are also provided for use as a medicament. More particularly, they are provided for use in the treatment of cancer. Most particularly, they are provided for use in the treatment of c-Met inhibitor resistant cancer.

As the role of c-Met is different in the tumor and the neutrophils (i.e., part of the stroma), methods to stratify patients in responders and non-responders are envisaged herein. Patients with high expression of MET in tumors and/or high expression of MET in stroma are likely to benefit from c-Met inhibition therapy, as a reduction in tumor c-Met is advantageous, and residual c-Met activity in neutrophils may be sufficient to ensure infiltration. Patients with low levels of Met in stroma are likely to experience adverse effects, as the cytotoxic effect of neutrophils on tumor cells is ablated upon further c-Met inhibition.

The methods that decrease granulocyte transmigration and/or recruitment by inhibiting the c-Met pathway are particularly suitable for treatment of inflammatory disease, particularly inflammatory disease with granulocyte (most particularly neutrophil) involvement. A specifically envisaged inflammatory disease with granulocyte involvement is asthma.

Accordingly, methods are provided to treat a subject with inflammatory disease (such as asthma), comprising administering a c-Met pathway inhibitor (such as a c-Met inhibitor) to the subject in need thereof.

It is particularly envisaged that at least part of the granulocytes in which the c-Met pathway is inhibited are neutrophils. Nevertheless, it is also envisaged that at least part of the granulocytes in which the c-Met pathway is inhibited are eosinophils, or even basophils.

According to a further aspect, compositions are provided for use as a medicament. Thus, a combination of a c-Met inhibitor with a granulocyte transmigration stimulating factor (most particularly a β2-integrin activator) is provided for use as a medicament. Most particularly, this combination is provided for use in the treatment of cancer.

According to particular embodiments, the c-Met inhibitor in these combinations is an antibody. For instance, the c-Met inhibitor can be the onartuzumab antibody. According to other (but non-exclusive) particular embodiments, the β2-integrin activator is an antibody. For instance, the β2-integrin activator may be the M18/2 antibody or a humanized version thereof. Other β2-integrin activating antibodies are known in the art, e.g., those described in Huang et al. (JBC, 275:21514-21524 (2000)) or in Ortlepp et al. (Eur. J Immunol., 25(3):637-43 (1995)).

According to other embodiments, a c-Met inhibitor is provided for use in treatment of asthma. Most particularly, the c-Met inhibitor is a c-Met inhibitory antibody. According to other embodiments, however, the c-Met inhibitor is a molecule that can be administered orally or nasally, to allow easier access to the airways and/or lungs of the subject to be treated.

All of these inhibitors or combinations may be provided as a pharmaceutical composition, comprising these ingredients and one or more pharmaceutically acceptable buffers or excipients.

### BRIEF DESCRIPTION OF THE DRAWINGS

FIG. 1. Met deletion in hematopoietic cells promotes cancer progression

a-b, Enhanced growth (a) and weight (b) of LLC tumors in WT mice transplanted with Tie2;Met^{lox/lox} bone marrow (BM) cells (KO→WT) compared to WT→WT mice (n=23-26).

c, Increased number of lung metastasis in LLC-tumor bearing KO→WT mice (n=23-26).

d-f, Quantification (d) and representative images (e, f) of TUNEL-stained LLC-tumor sections, showing reduced apoptosis in KO→WT mice (n=15-20).

g-i, Quantification (g) and representative images (h, i) of H&E-stained LLC-tumor sections, showing reduced necrosis (demarcated with a dotted line) in KO→WT mice (n=9).

j-l, Quantification (j) and representative images (k, l) of phosphohistone H3 (pHH3)-stained LLC-tumor sections, showing increased proliferation in KO→WT mice (n=9).

m, Enhanced growth of T241 tumors in KO→WT compared to WT→WT (n=8-9).

n, Enhanced growth of spontaneous mammary tumors in MMTV-PyMT mice transplanted with Met KO BM cells (KO→PyMT) compared to WT→PyMT mice (n=10-15).

o, Increased number of lung metastasis in KO→PyMT mice (n=10-15).

*, P<0.05 versus WT→WT in a-c, d, b, j, m; *, P<0.05 versus WT→PyMT in n. Scale bars denote 50 μm in e, f, k, 1; 100 μm in h, i. All graphs show mean±SEM.

FIG. 2. Met deletion in hematopoietic cells promotes tumor metastasis without affecting tumor vessel parameters

(a-c), Quantification (a) and representative images of H&E staining (b, c), showing increased pulmonary metastatic area (demarcated with black lines in b and c) in LLC-tumor bearing KO→WT compared to WT→WT mice (n=10).

(d), Increased metastatic index in LLC-tumor bearing KO→WT compared to WT→WT mice (n=23-26).

(e-h), Comparable CD31-positive vessel area (e), vessel density (f), lectin perfusion (g), and hypoxic (Pimo+) area (h) in LLC-tumors from WT→WT and KO→WT mice (n=6-8).

(i, j), Enhanced LLC tumor weight (i) and lung metastases (j) in Tie2;Met^{lox/lox} compared to Tie2;Met^{wt/wt} mice (n=10-12).

(k-o), Comparable tumor growth (k), CD31-positive vessel area (l), vessel density (m), lectin perfusion (n), and hypoxic (Pimo+) area (o) in endothelial cell specific MetKO (WT→KO) and control (WT→WT) mice (n=6).

(p), Comparable weight of Panc02 tumors in WT→WT and KO→WT mice (n=9-10).

*, P<0.05 versusTie2;Met^{wt/wt}; scale bar denotes 100 μm. All graphs show mean±SEM.

FIG. 3. Circulating and tumor-infiltrating immune cells upon Met deletion

(a-c), FACS analysis showing comparable percentages of circulating monocytes (a), lymphocytes (b) and neutrophils (c) in tumor-free or in LLC-tumor bearing WT→WT and KO→WT mice (n=7-12).

(d-i), Quantification of LLC-tumor sections stained for F4/80, NK1.1, CD45R, CD4, CD8 and CD11c, respectively, showing comparable infiltration of macrophages (d), natural killers (e), B lymphocytes (f), T helpers (g), cytotoxic lymphocytes (h) and dendritic cells (i) in WT→WT and KO→WT mice. (j) Quantification of Ly6G+ Panc02-tumor sections showing comparable neutrophil infiltration in WT→WT and KO→WT mice. (k) Quantification of LLC-tumor sections stained for F4/80 showing comparable infiltration of macrophages in LysM;Met^{lox/lox} and Lys;Met^{wt/wt} mice (n=4).

*, P<0.05 versus tumor free. All graphs show mean±SEM.

FIG. 4. Met deletion in hematopoietic cells impairs neutrophil infiltration to the tumor and metastatic niche

a-c, Quantification (a) and representative images (b, c) of Ly6G-stained LLC-tumor sections, showing reduced neutrophil infiltration in KO→WT mice at tumor endstage (n=7).

d, Quantification of Ly6G-stained LLC-tumor sections, showing reduced neutrophil infiltration in KO→WT mice at different time points of tumor progression.

e-f, Quantification of Ly6G-stained T241 or PyMT+ tumor sections showing reduced neutrophil infiltration in KO→WT (e) or in KO→PyMT (f) mice.

g, Quantification of Ly6G-stained lung sections showing comparable neutrophil infiltration in tumor-free mice (n=5) and reduced neutrophil infiltration in LLC-tumor bearing KO→WT mice (n=15).

h-i, Representative images of Ly6G-stained lung sections at tumor endstage.

j-k, Enhanced growth (j) and weight (k) of LLC tumors in LysM;Met^{lox/lox} compared to LysM;Met^{wt/wt} mice (n=9-10).

i, Quantification of Ly6G-stained LLC-tumor sections showing reduced neutrophil infiltration in LysM;Met^{lox/lox} mice (n=6-7).

m-n, Enhanced growth (m) and weight (n) of LLC tumors in nude mice transplanted with Met KO BM cells (KO→WT) compared to WT→WT mice (n=8-11).

o, Quantification of Ly6G-stained LLC-tumor sections, showing reduced neutrophil infiltration in KO→WT nude mice (n=7-10).

*, P<0.05 versus WT→WT in a, d, e, g, m, n, o; *, P<0.05 versus WT→PyMT in f; *, P<0.05 versus LysM;Met^{wt/wt} in j-l; #, P<0.05 versus day 9 or day 13 in d; #, P<0.05 versus tumor free in g; scale bar denotes 50 μm in b, c, h, i. All graphs show mean±SEM.

FIG. 5. Met deletion in hematopoietic cells does not influence neutrophil apoptosis but prevents neutrophil recruitment to the inflammatory site

a-b, Comparable intratumoral apoptosis of WT and Met KO neutrophils measured by immunohistochemistry (IHC; a; n=14) or FACS (b; n=6-7).

c-d, Quantification (c) and representative images (d) of Ly6G staining in ear-sections, showing reduced neutrophil infiltration in KO→WT mice upon phorbol ester (TPA)-induced cutaneous rash but not at baseline (CTRL; n=14-18).

e-f, Quantification of F4/80 (e) and CD3 (f) staining in ear-sections, showing comparable infiltration of macrophages and lymphocytes, respectively, upon TPA-induced cutaneous rash (n=15-23; n=6-8).

g, FACS analysis on peritoneal lavages showing reduced infiltration of neutrophils (but not macrophages) in KO→WT mice 4 hours after intra-peritoneal injection of sterile zymosan A (n=6).

*, P<0.05 versus WT→WT. #, P<0.05 versus CTRL. Scale bar denotes 100 μm. All graphs show mean±SEM.

FIG. 6. Met expression in neutrophils is induced by tumor-derived TNF-α or inflammatory stimuli

a-c, Q-PCR (a) and FACS (b, c) analysis showing induced MET expression in circulating neutrophils from LLC-tumor bearing WT mice and in tumor-associated neutrophils at both mRNA (a) and protein level (b, c; n=5).

d, Induction of MET expression in neutrophils sorted from human non-small cell lung tumors compared to neutrophils from healthy lung (n=4).

e-f, Induction of Met expression in circulating neutrophils from tumor-free WT mice after coculture with HUVEC pre-stimulated with IL-1, namely HUVEC (IL-1), but not with unstimulated HUVEC (e), or after stimulation with conditioned medium from LLC tumors (TCM) or cultured LLC (CCM) compared to mock medium (f) (n=4).

g, Q-PCR showing induction of MET expression in circulating human neutrophils after stimulation with conditioned medium from cultured A549 (A549-CM) (n=4).

h-i, Q-PCR showing induction of MET expression in circulating neutrophils from tumor-free WT mice (h) or in human neutrophils isolated from healthy volunteers (i) after stimulation with LPS or TNF-α (n=5).

j-k, Western blot analysis reveals induction of MET expression in BM neutrophils from tumor-free WT mice upon co-culture with HUVEC (IL-1), stimulation with TCM, CCM or TNF-a (j), and in human neutrophils isolated from healthy volunteers after stimulation with A549-CM, LPS or TNF-α (k).

l, RT-qPCR for c-Met mRNA in granulocytes (or polymorphonuclear cells, PMN), monocytes/macrophages (Mφ) and lymphocytes (Lc) sorted from the blood in tumor (TM) free or TM bearing WT mice or from TM in WT mice shows that c-Met RNA expression is strongly induced in tumor infiltrating granulocytes.

*, P<0.05 versus tumor free in a, c; *, P<0.05 versus healthy lung in d; *, P<0.05 versus mock in e, f, g, h, i. All graphs show mean±SEM.

FIG. 7. Hypoxia does not affect Met expression in neutrophils

(a, b), Comparable Met expression in mouse (a) or human (b) neutrophils cultured in normoxia or hypoxia. All graphs show mean±SEM.

FIG. 8. IL-1 potently induces TNF-α expression in ECs

Q-PCR showing Tnf-α induction in HUVEC upon stimulation with IL-1 compared to mock medium.

*, P<0.05 versus mock. Graph shows mean±SEM.

FIG. 9. Met induction in neutrophils is prevented by TNF-α blockade

a, Q-PCR for Met in mouse neutrophils, co-cultured with HUVEC or HUVEC (IL-1) transduced with shTNF-α or scramble as control, showing abolishment of Met induction upon TNF-α silencing in HUVEC (IL-1) (n=4).

b, Q-PCR showing abrogation of Met induction in mouse neutrophils co-cultured with HUVEC (IL-1) in presence of the TNF-α trap Enbrel; human IgG are used as control (n=4-5).

c-e, Q-PCR showing reduced Met expression in mouse neutrophils isolated from TNFRI KO mice when co-cultured with HUVEC (IL-1) (c), or stimulated with TNF-α (d) or TCM (e) compared to neutrophils isolated from WT or TNFRII KO mice (n=4).

f-g, Q-PCR showing abolishment of Met induction in mouse (f) or human neutrophils (g) when stimulated, respectively, with TCM or A549-CM in presence of the TNF-α trap Enbrel (n=4).

*, P<0.05 versus HUVEC scramble in a; *, P<0.05 versus human IgG in b, f; *, P<0.05 versus WT in c, d, e; *, P<0.05 versus A549-CM in g. #, P<0.05 versus mock in b, d-g; #, P<0.05 versus HUVEC in c. All graphs show mean±SEM.

FIG. 10: HGF-induced adhesion is mediated by β2-integrin

A, Granulocyte adhesion (% of Ly6G⁺ cells) in HGF-treated or non-stimulated (Mock) cells upon treatment with Rat IgG or a blocking β2-integrin antibody. B, Percentage of granulocytes bound to ICAM-1 in a soluble ICAM-1 binding assay, either non-treated, treated with Mg²⁺ as positive control or with HGF. C, Co-immunoprecipitation of active β2-integrin (through the binding to soluble ICAM-1) in non-stimulated cells and cells treated with HGF.

FIG. 11. MET is required for neutrophil transendothelial migration and cytotoxicity

a, b, FACS quantification of transmigrated neutrophils showing enhanced migration towards HGF (a) or TCM (b) of WT but not Met KO neutrophils (n=3); addition of the HGF trap decoy Met to TCM blunted TCM-induced transendothelial migration of WT neutrophils without affecting Met KO neutrophils (n=3-6).

c, FACS quantification of neutrophil adhesion to HUVEC (IL-1) showing increased adhesion in presence of HGF in WT but not Met KO neutrophils (n=3).

d, FACS quantification of neutrophil exudation into subcutaneous air pouches showing a strong migration of WT (but not Met KO) neutrophils towards HGF; CXCL1 was used as positive control of neutrophil migration (n=8-9).

e, Quantification of inducible nitric oxide synthase (Nos2) mRNA showing reduced expression levels in LLC-tumor-associated neutrophils sorted from KO→WT mice compared to WT→WT mice (n=10-12).

f, Quantification of nitric oxide (NO) production showing reduced NO level in medium conditioned by LLC tumors from KO→WT mice compared to WT→WT mice (n=8).

g-I, Quantification (g) and representative images (h, i) of LLC-tumor sections stained for 3-nitrotyrosine showing reduced tyrosine nitration in tumors grown in KO→WT mice compared to WT→WT mice (n=8-9).

j, Quantification of LLC cancer cell killing by neutrophils showing reduced cytotoxicity of Met KO neutrophils and ablation of WT neutrophil cytotoxicity in presence of nitric oxide synthase inhibitor L-NMMA (n=5).

k, FACS quantification of DAF-FM-positive neutrophils in co-culture with LLC cancer cells showing increased NO production in WT but not in Met KO neutrophils after HGF stimulation (n=10).

l, Quantification of LLC cancer cell killing by neutrophils showing increased cytotoxicity of WT (but not Met KO) neutrophils in response to HGF; the presence of L-NMMA abates this cytotoxicity in (n=5).

*, P<0.05 versus WT→WT; #, P<0.05 versus mock in a, b, c; #, P<0.05 versus PBS in d; #, P<0.05 versus (−) L-NMMA in j; #, P<0.05 versus (−) HGF in k; #, P<0.05 versus (−) HGF (−) L-NMMA in 1; $, P<0.05 versus (+) TCM (−) decoy Met in b; ; $, P<0.05 versus (+) HGF (−) L-NMMA in 1. All graphs show mean±SEM. Scale bar denotes 20 μm in h, i.

FIG. 12. MET does not affect neutrophil basal migration nor polarization

a, b FACS quantification of neutrophils migrated through naked porous filters (in absence of HUVEC) towards HGF (a) or TCM (b), showing comparable migration of WT and Met KO neutrophils (n=3). (c) FACS quantification of WT neutrophil adhesion to HUVEC pre-activated or not with IL-1, in presence or absence of HGF, showing increased adhesion to HUVEC (IL-1) only, in response to HGF. (d) Gene expression profile of LLC-tumor-associated neutrophils sorted from WT→WT or KO→WT mice (n=3-4). *, P<0.05 versus HUVEC (IL-1) mock; *, P<0.05 versus mock in b; *, P<0.05 versus HUVEC in c. All graphs show mean±SEM.

### DETAILED DESCRIPTION

### Definitions

The disclosure will be described with respect to particular embodiments and with reference to certain drawings but the disclosure is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g., “a” or “an,” “the,” this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the disclosure. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

The term “granulocyte(s),” as used in the application, refers to a category of white blood cells characterized by the presence of granules in their cytoplasm. A term used synonymously is polymorphonuclear (PMN) leukocyte. There are three types of granulocytes, distinguished by their appearance under Wright's stain: neutrophil granulocytes, which are the most abundant type, eosinophil granulocytes and basophil granulocytes. Neutrophils are recruited to the site of injury within minutes following trauma and are the hallmark of acute inflammation. Neutrophils comprise approximately 60% of blood leukocytes. During inflammation the number of neutrophils present in the blood dramatically increases. (As neutrophils are by far the most common type of granulocyte, many of the granulocyte effects are likely mainly neutrophil effects.) Neutrophils are highly phagocytic and form the first line of defense against invading pathogens, especially bacteria. They are also involved in the phagocytosis of dead tissue after injury during acute inflammation. Many of the defense mechanisms employed by neutrophils against pathogens, such as the release of granule contents and the generation of reactive oxygen species are pro-inflammatory and damaging to host tissue. In conditions characterized by excessive activation of neutrophils and/or impaired neutrophil apoptosis, chronic or persistent inflammation may result. Eosinophils comprise approximately 1-3% of blood leukocytes. Their primary role is in defense against parasites, in particular against helminthes and protozoal infection. In this regard, the cells comprise lysosomal granules containing cytotoxic compounds such as eosinophil cation protein, major basic protein, and peroxidase and other lysomal enzymes. Eosinophils are attracted by substances released by activated lymphocytes and mast cells. Although eosinophils may play a role in regulating hypersensitivity reactions by, for example, inhibiting mast cell histamine release degranulation, these cells may also damage tissue in allergic reactions. The cells accumulate in tissues and blood in a number of circumstances, for example, in hay fever, asthma, eczema, etc. As a result, through degranulation, they may contribute to or cause tissue damage associated with allergic reactions, for example, in asthma or allergic contact dermatitis. Basophils, which comprise less than 1% of circulating leukocytes, have deep blue granules that contain vasoactive substance and heparin. In allergic reactions, they are activated to degranulate, which may cause local tissue reactions and symptoms associated with acute hypersensitivity reactions.

As used herein, the term “transmigration” or “transendothelial migration” refers to the step in the leukocyte extravasation process wherein the leukocyte escapes the blood vessel, typically through gaps between endothelial cells (paracellular road). This step follows the rolling adhesion step on the inner vessel wall and the tight adhesion step. The process of blood vessel escape is also known as diapedesis.

“c-Met,” as used herein, refers to the gene encoding the hepatocyte growth factor (HGF) receptor, as well as to the encoded protein. The protein is a membrane receptor that possesses tyrosine kinase activity. It is also known as Met or the Met proto-oncogene (Gene ID: 4233 in humans). The “c-Met pathway” or “c-Met transmigration pathway,” as used herein, refers to the pathway triggered by c-Met signaling in granulocytes that results in transendothelial migration of the granulocytes. Upstream, this involves signaling of TNF-α through the TNFR1, which results in upregulation of c-Met. Downstream, this involves β2-integrin activation, which is induced by HGF signaling through c-Met. According to particular embodiments, the c-Met pathway does not involve the c-Met tyrosine kinase activity.

“β2-integrin,” sometimes also referred to as CD18, is part of the integrin beta chain family of proteins (Gene ID: 3689 in humans). Integrins are integral cell-surface proteins composed of an alpha chain and a beta chain. A given chain may combine with multiple partners resulting in different integrins. For example, beta 2 combines with the alpha L chain (also known as CD11a) to form the integrin LFA-1, and combines with the alpha M chain (also known as CD11b) to form the integrin Mac-1.

A “granulocyte-mediated inflammatory disease,” as used herein, refers to inflammatory diseases wherein granulocyte recruitment plays an important role in the disease process, e.g., because the release of granule contents and the generation of reactive oxygen species is damaging to the host tissue. According to particular embodiments, the granulocyte-mediated inflammatory disease is not cancer, or is not a neoplastic disease. According to other particular embodiments, the granulocyte-mediated inflammatory disease is a disease which is not caused by proliferation of leukocytes, for example, by abnormally excessive production of leukocytes. According to specific embodiments, the granulocyte-mediated inflammatory disease is a neutrophil mediated condition. Neutrophil mediated conditions for which the disclosure may find use include, but are not limited to, neutrophil mediated inflammatory conditions such as arthritis, pleurisy, lung fibrosis, systemic sclerosis, neutrophilic asthma and chronic obstructive pulmonary disease (COPD). According to alternative embodiments, the granulocyte-mediated inflammatory disease is an eosinophil mediated condition. These include, but are not limited to, asthma, atopic dermatitis, NERDS (nodules eosinophilia, rheumatism, dermatitis and swelling), hyper-eosinophilic syndrome or pulmonary fibrosis, contact dermatitis, eczema, and hay fever. According to alternative embodiments, the granulocyte-mediated inflammatory disease is a basophil mediated disease. Examples thereof include, but are not limited to, acute hypersensitivity reaction, asthma and allergies such as hay fever, chronic urticaria, psoriasis, and eczema.

It is shown herein that c-Met has an essential and previously unrecognized role in recruitment and transendothelial migration of granulocytes towards a site of tissue damage or infection (e.g., a tumor, a tissue confronted with chemicals or microbial compounds . . . ). This role is specific to granulocytes (particularly neutrophils), as c-Met deletion did not alter infiltration properties of other blood immune cells. Neutrophils are short-lived cells and key effectors of the innate immunity ²⁶. In response to chemotactic stimuli, neutrophils rapidly migrate from the bloodstream to inflammatory sites, thus, providing the first line of defense against host insults and pathogens. Similar to all the other cells belonging to the immune system, their plasticity and versatility in response to surrounding stimuli result in pro-tumoral or anti-tumoral phenotypes. Thus, neutrophils have been described to positively regulate tumor growth, angiogenesis, and metastasis ²⁷⁻³² or to restrain cancer cell proliferation and survival as well as metastatic seeding ^{28,33-36}.

Given the lack of knowledge on MET signaling in immune cells, we took advantage of a knockout mouse system deficient for MET in hematopoietic cells, which give origin to the immune system, in order to be able to dissect the function of this pleiotropic pathway in immune cells, and neutrophils in particular, during cancer progression.

We could show that MET promotes neutrophil cytotoxicity and chemoattraction in response to its ligand HGF. Genetic deletion of Met in myeloid cells enhances tumor growth and metastasis. This phenotype correlates with reduced neutrophil infiltration to both primary tumor and metastatic niche.

To extend the relevance of these findings in non-cancer settings, they were studied in models for inflammatory disease. There too, it was found that Met is required for neutrophil transudation during, e.g., skin rash or peritonitis.

Mechanistically, Met is induced by tumor-derived TNF-α or other inflammatory stimuli in both mouse and human neutrophils. This induction is instrumental for neutrophil transmigration across an activated endothelium and iNOS production upon HGF stimulation. Consequently, HGF/MET dependent nitric oxide release promotes neutrophil-mediated cytotoxicity and cancer cell killing, which abate tumor growth and metastasis. These findings disclose an anti-tumor role of MET in neutrophils and suggest a possible “Achilles' heel” of MET-targeted therapies.

In short, modulating c-Met levels and/or c-Met signaling offers a novel therapeutic approach to modulate transmigration and recruitment of granulocytes, and in particular neutrophils. This is particularly useful in diseases or situations characterized by excessive or insufficient granulocyte-mediated immune response.

Accordingly, methods are provided of modulating recruitment and transendothelial migration of granulocytes, comprising modulating the c-Met pathway in the granulocytes.

Modulating can be enhancing or inhibiting. Enhancing the c-Met pathway may refer to enhancing c-Met expression or activity. Enhancing expression may be achieved, e.g., using standard genetic engineering techniques to increase expression of c-Met. It is particularly envisaged that expression is enhanced in granulocytes, while not necessarily being enhanced in other cell types. Thus, expression may be driven by a promoter specific for the hematopoietic (e.g., Tie2 promoter, active in hematopoietic and endothelial cells) or myeloid (e.g., LysM promoter) lineage. Enhancing c-Met activity may be done by using c-Met agonists or mimetics, e.g., polypeptide agonists as described in EP2138508, c-Met agonistic antibodies (Bardelli et al., Biochem Biophys Res Commun. 334(4):1172-9, 2005), Magic-Factor 1 (Cassano et al., PLoS ONE 3(9): e3223, 2008), or small molecule agonists as described in, e.g., WO2010/068287.

Alternatively, the c-Met pathway may be enhanced by modulating upstream or downstream components of c-Met. For instance, administration of TNF-α will induce Met expression in granulocytes. This is, thus, an alternative way of increasing c-Met expression and activity. In diseases such as cancer, c-Met inhibition is envisaged as strategy.

Examples of cancer types, wherein c-Met is implicated and for which c-Met inhibition has been proposed as a therapeutic strategy include, but are not limited to, bladder carcinoma, breast carcinoma, cervical carcinoma, cholangiocarcinoma, colorectal carcinoma, endometrial carcinoma, esophageal carcinoma, gastric carcinoma, head and neck carcinoma, kidney carcinoma, liver carcinoma, lung carcinoma, nasopharyngeal carcinoma, ovarian carcinoma, pancreatic carcinoma, gall bladder carcinoma, prostate carcinoma, thyroid carcinoma, osteosarcoma, rhabdomyosarcoma, synovial sarcoma, Kaposi's sarcoma, leiomyosarcoma, fibrosarcoma, leukemia (AML, ALL, CML), lymphoma, multiple myeloma, glioblastoma, astrocytoma, melanoma, mesothelioma, and Wilm's tumor (Knudsen et al., Curr Opin Genet Dev. 2008; 18(1):87-96; Migliore et al., Eur J Cancer. 2008; 44(5):641-51; World Wide Web at vai.org/met).

As shown in the examples, inhibition of c-Met also may have pro-tumoral responses, explaining why some tumors exhibit resistance to c-Met inhibition. For these tumors, it may be beneficial to inhibit c-Met in the tumor environment, but to retain c-Met activity in granulocytes. Although this can be achieved by selective inhibition and stimulation of c-Met in the different tissues, it is often more practical to target a downstream effector of the c-Met pathway in granulocytes, so as not to interfere with c-Met inhibition in the tumor, while retaining granulocyte recruitment and transmigration. Thus, particularly in treatment of cancer, it is envisaged to enhance the c-Met pathway by enhancing its downstream effectors, as this allows dissociation of the c-Met mediated proliferation response (in the tumor) versus the c-Met mediated recruitment and transmigration (in the granulocytes). As shown in the examples, c-Met induced diapedesis is mediated by β2-integrin and HGF/c-Met signaling induces β2-integrin activation in granulocytes. Thus, increasing β2-integrin expression and/or activation in granulocytes has the same effect on transendothelial migration of granulocytes as enhancing c-Met, and it does not interfere with c-Met inhibitor activity in the tumor (as inhibitors target the enzymatic activity of the kinase). Accordingly, in particular embodiments, enhancing the c-Met transmigration pathway can be achieved by increasing β2-integrin expression and/or activation. Here also, expression can be increased by using standard genetic engineering techniques. Activation can be increased by using β2-integrin agonists or mimetics. Known β2-integrin agonists are antibodies, such as the M18/2 antibody (BD Biosciences; Driessens et al., J Leukoc Biol. 1996; 60(6):758-765), the KIM127 mAb (Stephens et al., Cell Adhes. Commun. 1995; 3: 375-384) which has been mapped to residues 413-575 in β2, in the middle third of the region C-terminal to the I-like domain, the CBR LFA-1/2 antibody (Petruzzelli et al., J. Immunol. 1995; 155: 854-866), the KIM185 antibody (Andrew et al., Eur. J. Immunol. 1993; 23: 2217-2222), or antibodies described in Huang et al. (JBC, 275:21514-21524 (2000)) or in Ortlepp et al. (Eur. J Immunol., 25(3):637-43 (1995)). Although the M18/2 antibody is a rat anti-mouse monoclonal antibody, it is within reach of the skilled person to make a humanized version, interacting with the human β2-integrin molecule.

Instead of antibodies, small molecules can be used as β2-integrin agonists or mimetics, such as those described by Yang et al. (J Biol Chem. 281(49):37904-12, 2006).

Alternatively, other granulocyte recruiting factors may be used. Indeed, the c-MET ligand HGF is one recruiting factor for granulocytes, but several other cytokines and chemokines are involved in chemotaxis and diapedesis as well. For instance, IL-8 (or CXCL-8), CXCL-1 (also known as KC in mice), interferon-gamma (IFN-γ), complement component 5a (C5a), leukotriene B4, G-CSF and IL-17 are all potent chemoattractants for granulocytes (particularly neutrophils). As shown in the examples section, TNF-α is also a very potent inducer of the MET pathway in neutrophils.

As can be deduced from the above, particularly when treating cancer, it is envisaged to simultaneously inhibit c-Met (in the tumor, to counter its proliferative effects) and enhance the (c-Met mediated) transmigration effect in granulocytes. Although it is envisaged to spatially separate the inhibitory and enhancing therapies (e.g., by restricting the therapies to a particular tissue or cell type, in casu tumoral tissue or granulocytes), it is often more practical to target different points in the pathway. Most particularly, it is envisaged to enhance only the c-Met mediated transmigration pathway, e.g., by increasing β2-integrin expression and/or activation, so as not to interfere with the antiproliferative cancer therapy. Alternatively, transmigration is enhanced in granulocytes by using granulocyte chemoattractants. In our experiments, we show that c-Met deficient granulocytes are indeed still responsive to, e.g., KC. Thus, it is envisaged that transmigration is enhanced by administering, e.g., KC, while at the same time inhibiting c-Met in the tumor.

Myriad c-Met inhibitors are known in the art, and many of them are being evaluated in clinical trials. Specific c-Met inhibitors include, but are not limited to, c-Met antibodies (e.g., onartuzumab, also known as MetMAb (Roche), ARGX-111 (arGEN-X)), c-Met nanobodies (e.g., as described in WO2012/042026), HGF antibodies (e.g., Rilotumumab (AMG102, Amgen), ficlatuzumab (SCH900105 or AV-299, AVEO pharmaceuticals), TAK-701 (Millennium)), small molecules directed to c-Met (e.g., AMG 337 (Amgen), AMG 208 (Amgen), tivantinib (ARQ197, ArQule), BMS-777607 (Bristol-Myers Squibb), EMD 1214063, EMD 1204831 (Merck Serono), INCB028060 (INC280, Incyte), LY2801653 (Eli Lilly), MK8033 (Merck), PF-04217903 (Pfizer), JNJ-38877605 (Johnson & Johnson)). There are also c-Met inhibitors that are less specific, i.e., that also inhibit other molecules or pathways than c-Met alone. They are also envisaged within the definition of c-Met inhibitors, since they inhibit c-Met. Examples include, but are not limited to, E7050 (Eisai), foretinib (XL880, GSK1363089, GlaxoSmithKline), amuvatinib (MP470, SuperGen), MGCD265 (MethylGene), MK2461 (Merck), crizotinib (PF-2341066, Pfizer), cabozantinib (XL184, Exelixis). Examples of c-Met inhibitors are also listed, e.g., in Table 1 of Liu et al., Trends Mol Med. 2010; 16(1):37-45; or in Gherardi et al., Nat Rev Cancer. 2012; 12(2):89-103, sections “HGF/SF and MET inhibitors for cancer therapy” and “Targeting HGF/SF-MET in cancer” from page 96-99.

As neutrophil-associated pro-tumorigenic effects are mainly dependent on TGF-β signaling and inhibition of TGF-β enables the N2, anti-tumoral, phenotype of neutrophils ³³, the combined administration of a c-Met inhibitors and a TGF-β inhibitor to a subject in need, thereof, is also envisaged herein. Different TGF-β inhibitors are described in the art and are commercially available. These include, but are not limited to, small molecule inhibitors such as A 83-01 (Tojo et al., Cancer. Sci. 96 791 (2005)), D 4476 (Callahan et al., J. Med. Chem. 45 999 (2002); GSK), GW 788388 (GSK), LY 364947 (Sawyer et al., J. Med. Chem. 46 3953 (2003)), RepSox (Gellibert et al., J. Med. Chem. 47 4494 (2004)), SB 431542 (GSK), SB 505124 (Byfield et al., Mol. Pharmacol. 65 744 (2004)), SB 525334 (GSK), SD 208 (Uhl et al., Cancer Res. 64 7954 (2004)), LY 2157299 (galunisertib), and LY 2109761; or inhibitory antibodies such as the TGF-β type II receptor antibody.

Combinations of c-Met inhibitors and TGF-β inhibitors are provided. They are also provided for use as a medicament. More particularly, they are provided for use in the treatment of cancer. Most particularly, they are provided for use in the treatment of c-Met inhibitor resistant cancer.

According to a further embodiment, according to this aspect, it is envisaged that combinations are provided of a c-Met inhibitor with a granulocyte transmigration stimulating factor, or pharmaceutical compositions containing such combinations. Particularly, envisaged granulocyte transmigration stimulating factors are β2-integrin activators, such as those listed above, e.g., the M18/2 antibody or a humanized version thereof. Particularly, envisaged c-Met inhibitors are those listed above, such as the MetMAb antibody.

These combinations or pharmaceutical compositions containing these combinations can be provided for use as a medicament. According to particular embodiments, they are provided for use in treatment of cancer. Typically, the pharmaceutical compositions will further comprise pharmaceutically acceptable excipients or carriers. These are well known to the skilled person.

The compositions provided for use in the treatment of cancer is equivalent to saying that methods are provided for the treatment of cancer, comprising administering a c-Met inhibitor and a β2-integrin activator to a subject in need thereof.

It is envisaged that the methods and combinations or compositions are particularly useful in the treatment of c-Met inhibitor resistant cancer.

Since neutrophils are the most common type of granulocytes and are part of the first-line responder inflammatory cells to migrate towards a site of inflammation, it is particularly envisaged that the granulocytes of which the recruitment and transmigration is enhanced are (at least in part, but up to all of the granulocytes) neutrophils.

As mentioned, the methods provided for modulating recruitment and transendothelial migration of granulocytes and comprising modulating the c-Met pathway in the granulocytes may also entail inhibiting the c-Met pathway in the granulocytes, thereby inhibiting recruitment and transendothelial migration. According to this aspect, methods are provided to decrease granulocyte recruitment and transmigration by inhibiting the c-Met pathway. This is particularly useful when a decrease in inflammatory response is desired, since prevention of transendothelial migration of granulocytes will lower the inflammatory leukocytes in the inflamed tissue. Accordingly, the methods are provided for treating inflammatory disease, particularly inflammatory disease with granulocyte involvement (i.e., granulocyte-mediated inflammatory disease).

A particularly well-known example of a disease characterized by excessive infiltration of granulocytes is asthma. Other examples of such diseases include, but are not limited to, adult respiratory distress syndrome (ARDS) (Craddock et al., N Engl J Med. 1977; 296(14):769-74), ischemia/reperfusion (I/R)-mediated renal, cardiac and skeletal muscle injury (Walden et al., Am J Physiol. 1990; 259(6 Pt 2):H1809-12), rheumatoid arthritis (Pillinger et al., Rheum Dis Clin North Am. 1995; 21(3):691-714), inflammatory bowel diseases such as Crohn's disease and ulcerative colitis (Wandall, Scand J Gastroenterol. 1985; 20(9):1151-6; Roberts-Thomson et al., Expert Rev Gastroenterol Hepatol. 2011; 5(6):703-16), allograft rejection (Surguin et al., Nephrol Ther. 2005; 1(3):161-6), transplantation (Marzi et al., Surgery. 1992; 111(1):90-7) and eosinophilic diseases that typically affect the upper and lower airways, skin and gastrointestinal tract (see list further). According to a very specific embodiment, the disease characterized by excessive infiltration of granulocytes is not rheumatoid arthritis.

Thus, methods are provided to treat diseases characterized by excessive recruitment and/or infiltration of granulocytes by inhibiting the c-Met pathway, particularly by inhibiting the c-Met pathway in the granulocytes.

It is particularly envisaged to inhibit the c-Met pathway by inhibiting expression and/or activity of c-Met. Indeed, many c-Met inhibitors are known, as already described earlier. These c-Met inhibitors can be used to inhibit the c-Met pathway and, thus, decrease the recruitment and transmigration of granulocytes. A particularly envisaged inhibitor is the onartuzumab (MetMAb) antibody.

In other words, these c-Met inhibitors can be used to treat diseases characterized by excessive recruitment and/or infiltration of granulocytes, particularly those listed above, such as asthma.

To the best of our knowledge, c-Met inhibitors, thus far, have only been evaluated in cancer, and no other diseases have been linked with excess c-Met signaling. This is the first time that c-Met inhibitors are proven useful in the treatment of inflammatory disease.

Accordingly, c-Met inhibitors, such as, e.g., c-Met inhibitory antibodies, are provided for use in treatment of inflammatory disease. More particularly, c-Met inhibitors are provided for use in treatment of inflammatory disease with granulocyte involvement, i.e., for diseases characterized by excessive recruitment and/or infiltration of granulocytes. A most particularly envisaged disease in this context is asthma.

Although neutrophils are the most common granulocytes, and it is envisaged that at least part of the granulocytes whose transmigration is decreased are neutrophils, this does not mean that c-Met should not be inhibited in other granulocytes. For instance, it is well known that eosinophils play an important role in the pathogenesis of asthma (Uhm et al., Allergy Asthma Immunol Res. 2012; 4(2):68-79). Other examples of eosinophilic disease include, but are not limited to, eosinophilic esophagitis, eosinophilic gastritis, eosinophilic gastroenteritis, eosinophilic colitis, eosinophilic fasciitis, eosinophilic pneumonia, eosinophilic cystitis, Churg-Strauss syndrome and hypereosinophilic syndrome.

Thus, particularly in the treatment of these diseases, it is envisaged that at least part of the granulocytes in which the c-Met pathway is inhibited are eosinophils.

It is to be understood that although particular embodiments, specific configurations as well as materials and/or molecules, have been discussed herein for cells and methods according to the disclosure, various changes or modifications in form and detail may be made without departing from the scope and spirit of this disclosure. The following examples are provided to better illustrate particular embodiments, and they should not be considered limiting the application. The application is limited only by the claims.

### Examples

### Material and Methods

Animals: The Met^{lox/lox} mice were a gift of Dr. Thorgeirsson (Center for Cancer Research, NCI, Bethesda, Md.). The Tie2:Cre, LysM:Cre and MMTV-PyMT transgenic lines were obtained from our mouse facility. C57BL/6 mice and C57BL/6 nude mice were purchased from Harlan and from Taconic, respectively. TNFRI KO mice and TNFRII KO mice were a gift of Dr. Libert (VIB Department for molecular biomedical research, UGent). All the experimental procedures were approved by the Institutional Animal Care and Research Advisory Committee of the K.U. Leuven.

Bone marrow transplantation: recipient mice were lethally irradiated (9.5 Gy) and then intravenously injected with 107 BM cells from Tie2;Metlox/lox or Tie2;Metwt/wt mice. Tumor experiments were initiated 5 weeks after BM reconstitution. Blood cell count was determined using a hemocytometer on peripheral blood collected by retro-orbital bleeding.

Tumor models: 2×10⁶ Lewis lung carcinoma (LLC) or T241 fibroscarcoma cells were injected subcutaneously. Tumor volumes were measured 3 times a week with a calliper. 10⁶ Panc02 cells were orthotopically injected in the head of the pancreas. 21 days after injection for LLC and T241, or 10 days after injection for Panc02, tumors were weighed and collected for histological examination. Lung metastases were contrasted by intratracheal injection of a 15% India ink solution or by hematoxylin eosin (H&E) staining on lung paraffin sections.

Adhesion Assay: 4×10⁴ HUVEC were seeded in M199 20% FBS in 96-multiwell previously coated with 0.1% gelatin. After 12 h, HUVEC were stimulated with 5 ng/ml IL-1 in DMEM 10% FBS at 37° C. After 4 hours the endothelial monolayer was thoroughly washed and 2.5×105 WBC were seeded on top, with or without murine HGF (50 ng/ml). After 15′ non-adherent cells were washed out whereas adherent cells were detached by using Cell Dissociation Buffer, Enzyme Free, PBS-Based (Gibco). Cells were stained with Ly6G-APC, washed and resuspended in PBS-BSA 0.1% with unlabeled counting beads (BD Bioscience) and quantified with FACS Canto II (BD Bioscience).

Transmigration and Migration Assay: For the transmigration assay, 2×10⁵ HUVEC were seeded on 3 μm polycarbonate membrane (Transwell; Costar) previously coated with 0.1% gelatin in M199 20% FBS. After 12 h, HUVEC were stimulated for 4 hours at 37° C. in DMEM 10% FBS with 5 ng/ml IL-1 and then washed. 5×105 WBC were seeded on top of the endothelial monolayer, while mock medium (+/− decoy Met), TCM (+/− decoy Met) or 50 ng/ml murine HGF was added in the bottom. After 2 hours at 37° C., transmigrated cells were collected from the lower chambers and from the bottom part of the filter with cold PBS 0.5% EDTA. Cells were stained and Ly6G+ cells quantified as above. In the migration assays WBC were seeded directly on top of 3 μm polycarbonate porous membranes.

Cytotoxicity assay: LLC-shMet were transduced with a luciferase-expressing lentivirus (EXhLUC-Lvl 14 from GeneCopoeia); 104 LLC were seeded in DMEM 10% FBS in 96-multiwell. After 4 h, 0.2×10⁶ neutrophils purified from the blood of LLC-tumor bearing mice or sorted from LLC-tumors were co-cultured with the LLC in DMEM 2% FBS for 4 hours at 37° C., with or without 100 ng/ml HGF or 1 mM L-NMMA (SIGMA). After washing, adherent cells were lysate in 0.2% Tryton 1 mM DTT. Luciferase signal was revealed with a microplate luminometer.

Cell lines: murine Lewis lung carcinoma cells (LLC) were obtained from American Type Culture Collection (ATCC) and cultured in DMEM (Gibco) supplemented with 2 mmol/L glutamine, 100 units/ml penicillin, 100 μg/ml streptomycin and containing 10% FBS. The murine pancreatic tumor cell line Panc02 and the murine fibrosarcoma cell line T241 were cultured in RPMI (Gibco) supplemented with 2 mmol/L glutamine, 100 units/ml penicillin, 100 μg/ml streptomycin and containing 10% FBS. Human non-small cell lung carcinoma A549 cells were cultured in DMEM supplemented with 2 mmol/L glutamine, 100 units/ml penicillin, 100 μg/ml streptomycin and containing 10% FBS. Human Umbilical Vein Endothelial Cells (HUVEC) were isolated from human umbilical cords and maintained in M199 (Invitrogen) supplemented with 20% FBS, 2 mmol/L glutamine, 100 units/ml penicillin, 100 μg/ml streptomycin, 0.15% Heparin, 20 μg/ml ECGS (M199 complete). 0.1% pork gelatin was used to favor the adhesion of HUVEC to the flask bottom. Lentiviral vectors containing short hairpin RNA were bought from SIGMA and used to produce lentivirus in 293T-HEK cells and transduce LLC to silence Met (LLC shMet) or HUVEC to silence TNF-α (HUVEC shTNF-α). Scramble lentiviral vectors were used as control. Transduced cells were selected with 8 μg/ml puromycine. All cells were maintained in a humidified incubator in 5% CO2 and 95% air at 37° C.

| shRNA | TRC number | Sequence |
|---|---|---|
| Human | TRCN0000003757 | CCGGCTGTAGCCCATGTTGTAGCAA |
| Tnfα | | CTCGAGTTGCTACAACATGGGCTAC |
| | | AGTTTTT (SEQ ID NO: 1) |
| Mouse | TRCN0000023529 | CCGGCGGGATTCTTTCCAAACACTT |
| MET | | CTCGAGAAGTGTTTGGAAAGAATCC |
| | | CGTTTTT (SEQ ID NO: 2) |
| Mouse | TRCN0000023530 | CCGGGCACGACAAATACGTTGAAAT |
| Met | | CTCGAGATTTCAACGTATTTGTCGT |
| | | GCTTTTT (SEQ ID NO: 3) |
| Scramble | SHC002V | CCGGCAACAAGATGAAGAGCACCAA |
| | | CTCGAGTTGGTGCTCTTCATCTTGT |
| | | TGTTTTT (SEQ ID NO: 4) |

Mouse White Blood Cell (WBC) isolation: blood was collected from the retro-orbital vein in 10% heparin. For WBC purification, the blood was diluted in dextran 1.25% in saline solution to allow the sedimentation of red blood cells (RBC). After 30′, the supernatant was collected and washed in PBS-BSA 0.1%. The remaining RBC were lysed in a hypotonic solution of NaCl 0.2% for 30″ and brought in isotonic condition with NaCl 1.6%. WBC were washed in PBS-BSA 0.1%, counted and resuspended according to the experimental setting.

Mouse Blood Neutrophil isolation: blood was collected from the retro-orbital vein in 10% heparin and diluted in an equal volume of PBS-BSA 0.5%. Up to 5 ml of diluted blood was layered on top of a discontinuous gradient of Histopaque 1119 (4 ml) and Histopaque 1077 (5 ml) from SIGMA. The gradient was centrifuged for 30′ at 700 g with the brake off. The neutrophil layer between the Histopaque 1077 and 1119 was collected and washed in PBS-BSA 0.5%. RBC lysis was performed as described; neutrophils were washed in BSA 0.5%, counted and resuspended according to the experimental condition. For RNA isolation, blood was sedimented in dextran 1.25% in saline solution and neutrophils were purified with a negative selection with magnetic beads ⁵¹. For both protocols, neutrophil purity by hemocytometer assessment was higher than 95%.

Bone marrow neutrophil isolation: in order to reach reasonable amount of protein, all the Western Blot analyses in mice were performed on neutrophils isolated from bone marrows. Mice were sacrificed by cervical dislocation. Femurs and tibias were isolated and collected in cold sterile Hank Balanced Salt Solution (HBSS, Invitrogen) with 0.5% BSA. Bone marrow cells were collected by flushing the bones with HBSS-0.5% BSA. Cells were layered on top of 3 ml Nycoprep 1.077A (Axis Shield). Mononuclear cells were, therefore, isolated and removed. The pellet of neutrophils and RBCs was washed in PBS and RBC lysis and was performed as described. Neutrophils were washed again, counted and resuspended according to the experimental setting. Neutrophil purity by hemocytometer assessment was higher than 85%.

Human neutrophil isolation: 10 ml of venous blood from healthy volunteers were collected in citrate-coated tubes and isolated by erythrocyte sedimentation with dextran and purification with a discontinuous plasma-Percoll gradient as already described ⁵².

FACS analysis and flow sorting of mouse blood or tumor-associated cells: blood was collected in 10% heparin and stained for 20 minutes at room temperature. After RBC lysis, cells were washed and resuspended in FACS buffer (PBS containing 2% FBS and 2 mM EDTA). Tumors were minced in RPMI medium containing 0.1% collagenase type I and 0.2% dispase type I (30 minutes at 37° C.), passed through a 19 G needle and filtered. After RBC lysis, cells were resuspended in FACS buffer (PBS containing 2% FBS and 2 mM EDTA) and stained for 20 minutes at 4° C. Cells were analyzed with FACS Canto II (BD Bioscience). The following antibodies were used: anti-Ly6G (1A8), CD45, CD11b, AnnexinV (all from BD-Pharmingen), Met, CD115, CD11c (all from eBioscience). For tumor-associated neutrophil sorting, myeloid population was enriched by coating with CD11b-conjugated magnetic bead (MACS milteny) and separation through magnetic column (MACS milteny), stained with Ly6G and sorted with FACS Aria I (BD Bioscience). Cells were collected in RLT for RNA extraction or resuspended according to the experimental conditions.

Lung cancer patients: we enrolled 4 non-small cell lung carcinoma-patients; exclusion criteria were history of oncological, chronic inflammatory and autoimmune diseases within 10 years prior to this study. All participants gave written informed consent. Flow sorting of human tumor- or tissue-associated neutrophils from lung cancer patients: lung tumor biopsies and healthy tissue were minced in RPMI medium containing 0.1% collagenase type I, 0.2% dispase type I and DNase I 100 U/ml (60 minutes at 37° C.), passed through a 19 G needle and filtered. After RBC lysis, cells were resuspended in FACS buffer (PBS containing 2% FBS and 2 mM EDTA) and counted. Myeloid population was enriched by coating with CD11b-conjugated magnetic beads (MACS milteny) and separation through magnetic column (MACS milteny), stained with anti-CD66b APC (BD Pharmingen) for 20′ on ice and sorted with FACS Aria I (BD Bioscience). Cells were counted and resuspended in RLT for RNA extraction.

TPA model of acute skin inflammation: phorbol ester TPA was used to induce acute skin inflammation as described before. Briefly, TPA (2.5 μg in 20 μl acetone per mouse) was topically applied to the left outside ear of anaesthetized mice. The right ear was painted with acetone alone as a carrier control. Mice were sacrificed after 24 hours and the ear collected in 2% PFA for histological analysis.

Zymosan-mediated acute peritonitis model: to induce acute peritonitis, zymosan A (Sigma) was prepared at 2 mg/ml in sterile PBS; 0.1 mg/mouse was injected intra-peritoneum in BMT mice. After 4 hours, mice were sacrificed and inflammatory cells were harvested by peritoneal lavage with 2 ml of PBS. Cells were counted with a Burker chamber and stained for Ly6G and F4/80 for FACS analysis.

Air Pouch Assay: to create subcutaneous air pouches, bone marrow transplanted WT and KO mice were injected with 3 ml of sterile air by dorsal subcutaneous injection with a butterfly 23G needle on day 0 and on day 3. On day 6, 200 ng/mouse of CXCL1 or murine HGF in 0.5 ml PBS-Heparin or PBS-Heparin as control, were injected in the dorsal camera created with the previous injection. After 4 hours, inflammatory cells were harvested by washing the pouch with 8 ml of PBS. Cells were stained with Ly6G-APC, washed and resuspended in PBS-BSA 0.1% with unlabeled counting beads and quantified with FACS Canto II (BD Bioscience).

Histology and immunostainings: for serial 7-μm-thick sections, tissue samples were immediately frozen in OCT compound or fixed in 2% PFA overnight at 4° C., dehydrated and embedded in paraffin. Paraffin slides were first rehydrated to further proceed with antigen retrieval in citrate solution (DAKO). Cryo-sections were thawed in water and fixed in 100% methanol. If necessary, 0.3% H2O2 was added to methanol to block endogenous peroxidases. The sections were blocked with the appropriate serum (DAKO) and incubated overnight with the following antibodies: rat anti-Ly6G (BD-Parmingen clone 1A8) 1:100, rat anti-CD31 (BD Pharmingen) 1:200, rabbit anti-FITC (Serotec) 1:200, goat anti-phosphohistone H3 (pHH3) (Cell Signaling) 1:100, rat anti-F4/80 (Serotec) 1:100, mouse anti-NK1.1-biotin (BD Pharmingen) 1:200, rat anti-CD45R (BD Pharmingen) 1:100, rat anti-CD4 (BD Pharmingen) 1:100, rat anti-CD8 (BioXCell clone 53-6.72) 1:100, hamster anti-CD11c biotin (eBioscience) 1:100, mouse anti-3-nitrotyrosin 1:200 (Santa Cruz). Appropriate secondary antibodies were used: Alexa488- or Alexa568-conjugated secondary antibodies (Molecular Probes) 1:200, HRP-labeled antibodies (DAKO) 1:100. When necessary, Tyramide Signaling Amplification (Perkin Elmer, Life Sciences) was performed according to the manufacturer's instructions. Whenever sections were stained in fluorescence, ProLong Gold mounting medium with DAPI (Invitrogen) was used. Otherwise, 3,3′-diaminobenzidine was used as detection method followed by Harris' haematoxilin counterstaining, dehydration and mounting with DPX. Apoptotic cells were detected by the TUNEL method, using the AptoTag peroxidase in situ apoptosis detection kit (Millipore) according to the manufacturer's instructions. Tumor necrosis and lung metastasis were evaluated by H&E staining. Microscopic analysis was done with an Olympus BX41 microscope and CellSense imaging software or a Zeiss Axioplan microscope with KS300 image analysis software.

Hypoxia assessment and tumor perfusion: tumor hypoxia was detected by injection of 60 mg/kg pimonidazole hydrochloride into tumor-bearing mice one hour before tumors harvesting. To detect the formation of pimonidazole adducts, tumor cryosections were immunostained with Hypoxyprobe-1-Mab1 (Hypoxyprobe kit, Chemicon) following the manufacturer's instructions. Perfused tumor vessels were counted on tumor cryosections from mice injected intravenously with 0.05 mg FITC-conjugated lectin (Lycopersicon esculentum; Vector Laboratories).

Tumor Conditioned Medium (TCM) and LLC (or A549) conditioned medium (CCM) preparation: end-stage LLC tumor explants from WT mice were homogenized and incubated at 37° C. in DMEM (supplemented with 2 mmol/L glutamine, 100 units/ml penicillin/100 μg/ml streptomycin) FBS-free. 2×104 LLC (or A549) were seeded in 6-multiwell in DMEM 10% FBS and incubated at 37° C. Medium alone was used to prepare mock controls. After 72 hours, the medium was filtered, supplemented with 2 mmol/L glutamine and 20 mM HEPES and kept at −20° C. TCM and mock 0% were diluted 1:5 in DMEM 10% FBS; CCM and mock 10% were diluted 4:5 in DMEM FBS-free.

Western blot: 2×10⁶ bone marrow neutrophils from WT mice were stimulated with TCM, CCM, 100 ng/ml of murine TNF-α (or mock medium 0% FBS or 10% FBS as control) for 20 hours at 37° C. For the co-culture with HUVEC, a monolayer of HUVEC was stimulated for 4 hours with 5 ng/ml IL-1 at 37° C., and washed before neutrophil seeding. After 20 hours of stimulation, neutrophils were collected using Cell Dissociation Buffer, Enzyme Free, PBS-Based (Gibco). Cells were washed in PBS, lysed in 15 μl of a protease inhibitor mixture and incubated for 15 minutes on ice. The stock solution was obtained by dissolving one tablet of Complete Mini protease inhibitor mixture (CI, Roche) in 5 ml of PBS with 2 mM diisopropyl fluorophosphate (DFP; Acros Organics, Morris Plains, N.J.). After addition of an equal amount of 2×SDS sample buffer supplemented with 4% 2-mercaptoethanol, the lysates were boiled for 15 minutes and kept at −80° C. until use. 30×10⁶ neutrophils purified from healthy volunteer blood and stimulated with A549-CM, 100 ng/ml of human TNF-α, 50 ng/ml LPS (or mock medium 10% FBS as control) for 20 hours. Cells were incubated with DFP 2.7 mM for 15′ at 4° C., collected and washed in PBS, DFP 2.7 mM, CI 1X, and lysed in hot Laemlii buffer (25% SDS 10%, 25% Tris-HCl pH 6.8) at 96° C. for 10′. Cell lysates were sonicated, cleared and quantified. 6× loading buffer was added before loading on the gel. The following antibodies were used: mouse anti-mouse Met (clone 3D4; Invitrogen), mouse anti-mouse β-actin (Santa Cruz), rabbit anti-human Met (clone D1C2-XP; Cell Signaling), HRP-conjugated anti-beta-tubulin (Abcam). Signal was visualized by Enhanced Chemiluminescent Reagents (ECL, Invitrogen) or West Femto by Thermo Scientific according to the manufacturer's instructions.

Quantitative RT-PCR: for mRNA analysis, 1×10⁵ or 3×10⁵ mouse or human neutrophils, respectively, were incubated in normoxic (21% oxygen) or hypoxic condition (1% oxygen) or stimulated with TCM (plus 50 μg/ml Enbrel or human IgG when indicated), CCM, A549-CM, 100 ng/ml of murine or human TNF-α, 50 ng/ml LPS, or mock medium in 96-multiwell for 4 hours at 37° C. 2×10⁵ HUVEC were seeded in 24-multiwell coated with 0.1% gelatin and stimulated with 5 ng/ml IL-1 in DMEM 10% FBS for 4 hours at 37° C. Cells were washed in PBS, collected in RLT buffer (QIAGEN®) and kept at −80° C. RNA was extracted with an RNase Micro kit (QIAGEN®) according to manufacturer's instructions. Reverse transcription to cDNA was performed with the SuperScript III Reverse Transcriptase (Invitrogen) according to manufacturer's protocol. Pre-made assays were purchased from Applied Biosystem, except for Nos2 that was provided by IDT. cDNA, preferential primers and the TAQMAN® Fast Universal PCR Master Mix were prepared in a volume of 10 μl according to manufacturer's instructions (Applied Biosystems). Samples were loaded into an optical 96-well Fast Thermal Cycling plate (Applied Biosystems), followed by qRT-PCR in an Applied Biosystems 7500 Fast Real-Time PCR system.

Decoy Met preparation: HEK 293T cells were transfected with a lentiviral vector expressing Decoy Met 14. Medium was changed after 14 hours and collected after 30 hours and then filtered. 20 mM hepes and anti-flag M2 affinity gel (Sigma) were added to the medium; after an overnight incubation on a wheel at 4° C., Decoy Met bound to the resine was washed 3 times in TBS, and eluted by incubation with 50 ng/μl of flag peptide (SIGMA) for 45′ at 4° C. Quantification was done by running 10 μl of purified Decoy Met on a 10% polyacrylamide gel together with known amount of BSA followed by Comassie staining. Decoy Met (or flag peptide as control) was used at 0.5 ng/μl after 10′ pre-incubation with mock or TCM or 459-CM at 37° C.

Tumor-derived nitric oxide production: LLC tumors were collected 8 days after injection, cut in pieces of about 5×5 mm, weighted and incubate at 37° C. in 24-multiwell with 800 μl of DMEM. After 24 hours, the media was collected, centrifuged to remove cell debris, and NO levels were measured using the Greiss reagent system kit (Promega).

Nitric oxide measurement by FACS: neutrophils isolated from the blood of WT or KO LLC tumor bearing mice were co-cultured for 4 hours with LLC shMet, washed, and resuspended in PBSHepes 20 mM, incubated for 10′ with 5 μM DAF-FM diacetate (Molecular probes) in the absence or presence of HGF (100 ng/ml) at 37° C., washed and analyzed by FACS.

Statistics: Data indicate mean±SEM of representative experiments. Statistical significance was calculated by two-tailed unpaired t-test for two data sets, with p<0.05 considered statistically significant.

### Example 1

### Generation of Lineage-Specific c-Met Deficient Mice and Effect on Tumor Growth

To study the in vivo function of MET in immune cells, we generated conditional knockout mice lacking Met in the hematopoietic lineage ³⁷. We intercrossed Met floxed mice with Tie2:Cre mice, which delete floxed genes in both hematopoietic and endothelial cells ³⁸, thus generating Tie2;Metlox/lox or Tie2;Metwt/wt mice as controls. Tie2;Metlox/lox mice developed normally, were fertile, had normal body weights, and exhibited no obvious organ defects upon macroscopic inspection or histological analysis (not shown). Blood counts were comparable in both genotypes (Table 1).

TABLE 1
Blood count in Tie2;Met^{wt/wt} or Tie2;Met^{lox/lox} tumor-free mice.

| Tumour free | Tier2;Met^{wt/wt} | Tie2;Met^{lox/lox} |
|---|---|---|
| WBC (k/μl) | 5.68 ± 1.44 | 5.55 ± 1.29 |
| NEU (%) | 23.03 ± 5.45 | 29.67 ± 7.88 |
| LYM (%) | 69.72 ± 6.46 | 72.03 ± 4.89 |
| MON (%) | 1.24 ± 0.37 | 2.86 ± 1.15 |
| EOS (%) | 0.12 ± 0.05 | 0.17 ± 0.12 |
| BAS (%) | 3.38 ± 1.32 | 4.47 ± 2.1 |
| RBC (M/μl) | 5.21 ± 1.91 | 4.89 ± 1.52 |
| HCT (%) | 71.3 ± 3.43 | 60.2 ± 13.38 |
| MCHC (g/dl) | 15.83 ± 2.65 | 18.3 ± 0.26 |
| PLT (K/μl) | 439.73 ± 26.64 | 508 ± 55.79 |

To ensure specific deletion of Met in the hematopoietic lineage only, we reconstituted lethally irradiated wild-type (WT) mice with bone marrow (BM) cells from Tie2;Met^{wt/wt} (Met WT) or Tie2;Met^{lox/lox} (Met knockout; KO) mice, producing WT→WT or KO→WT mice, respectively.

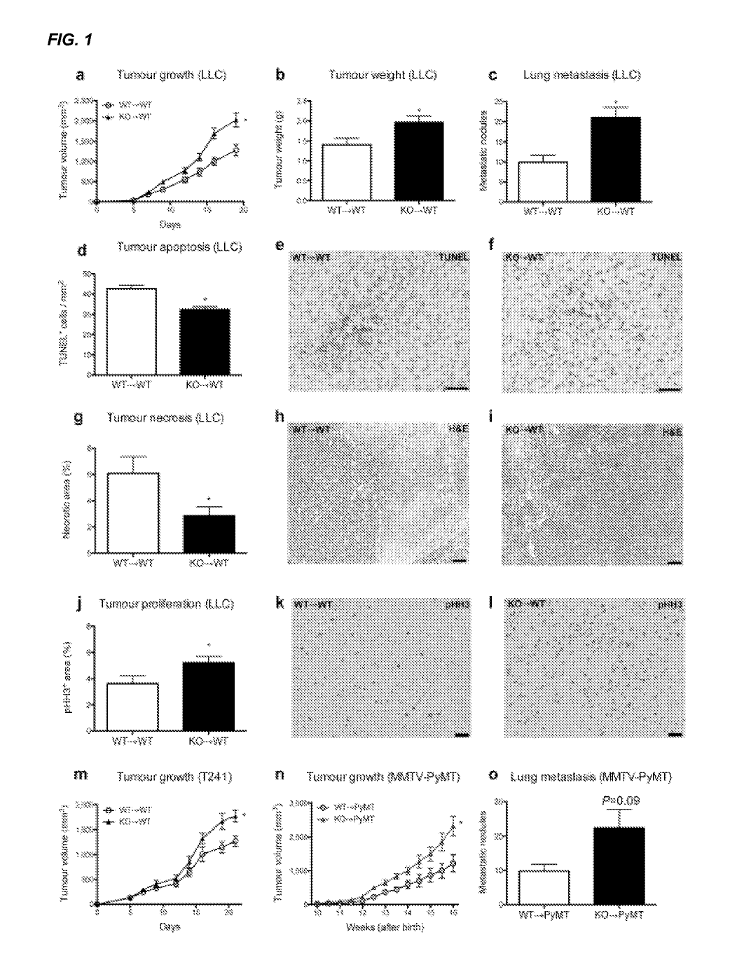

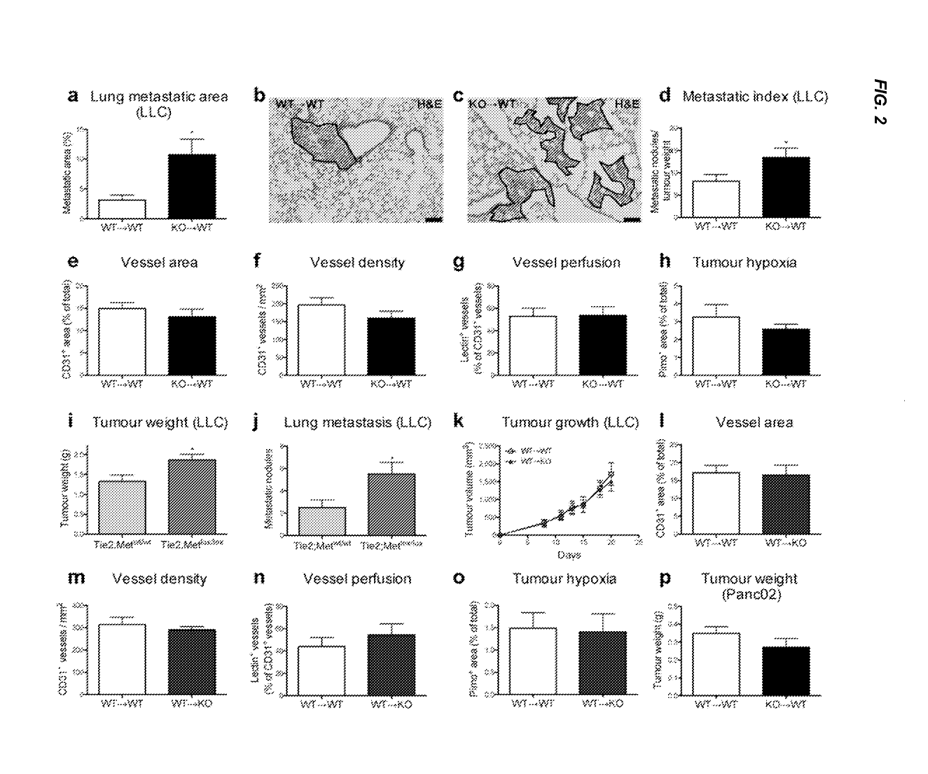

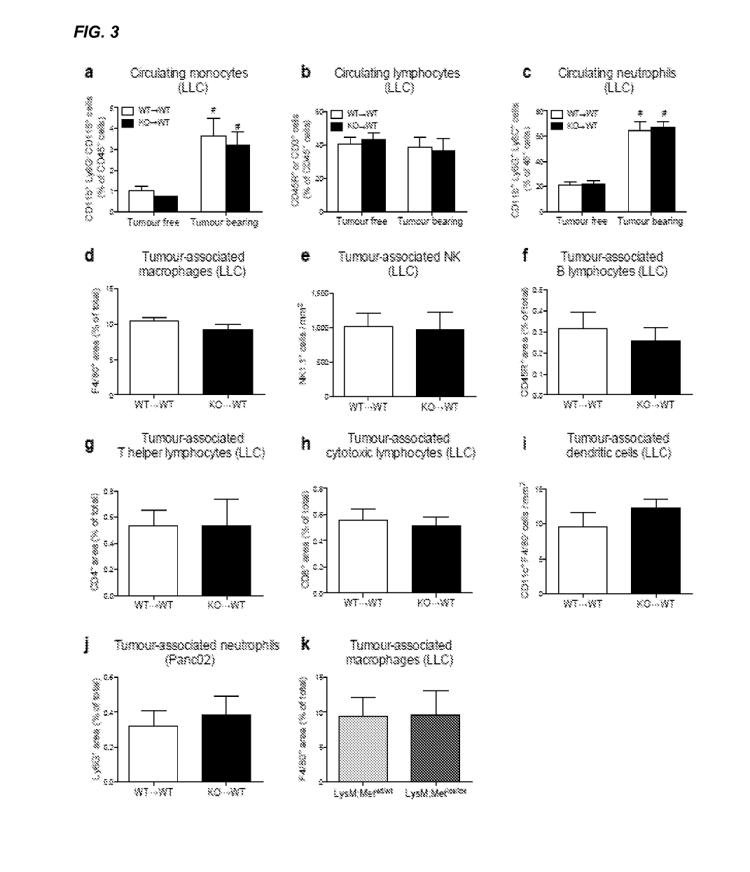

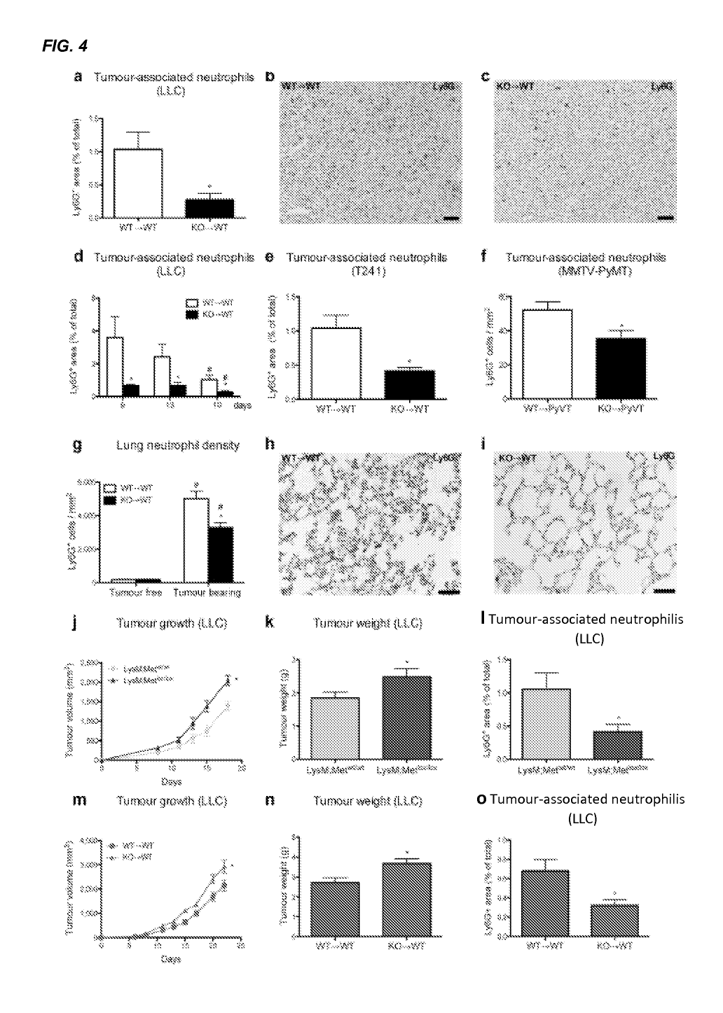

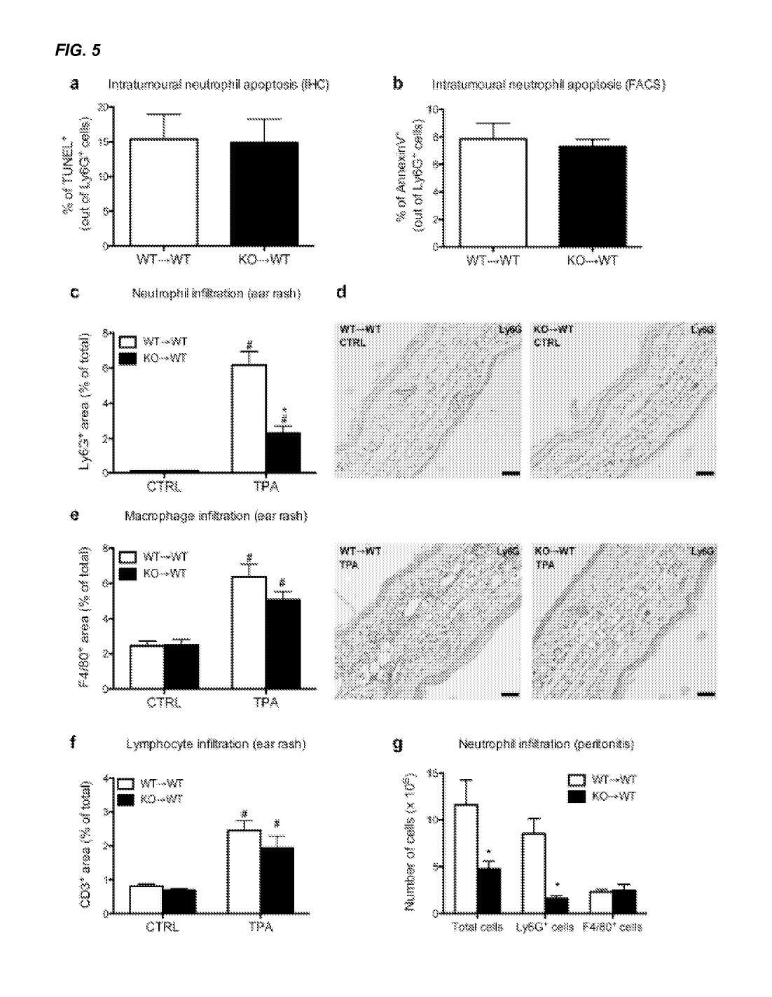

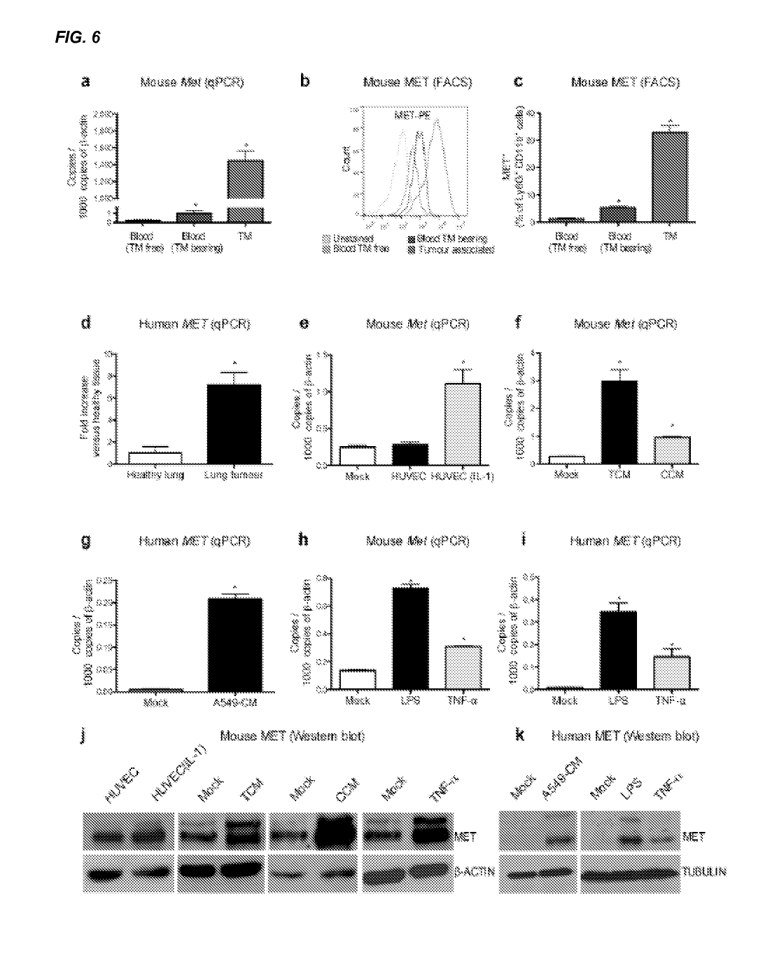

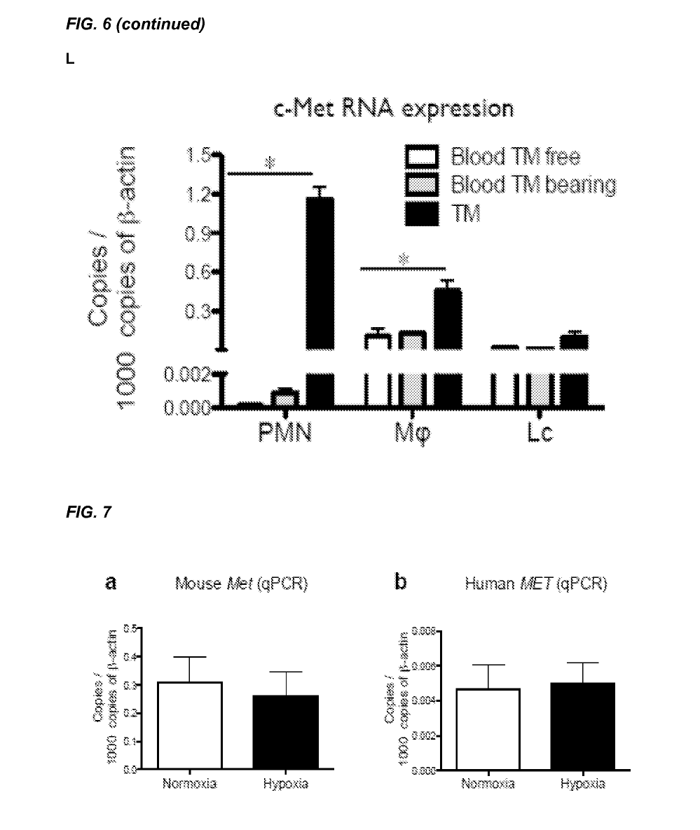

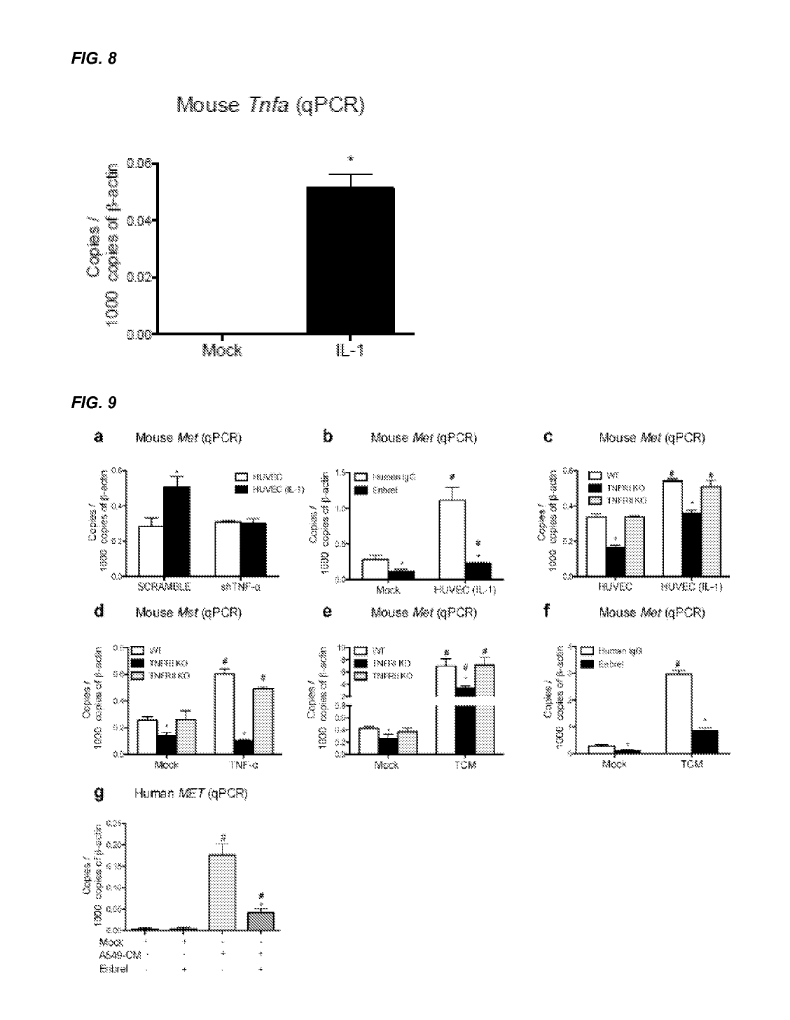

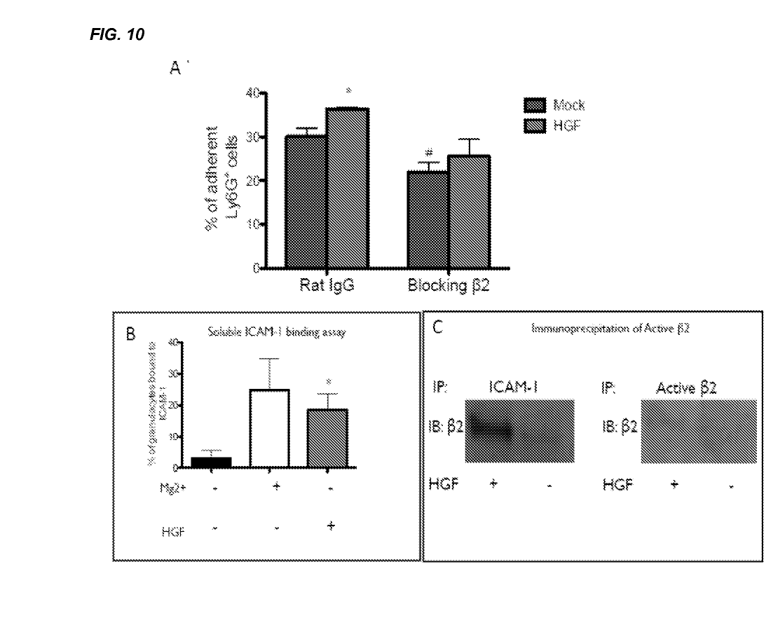

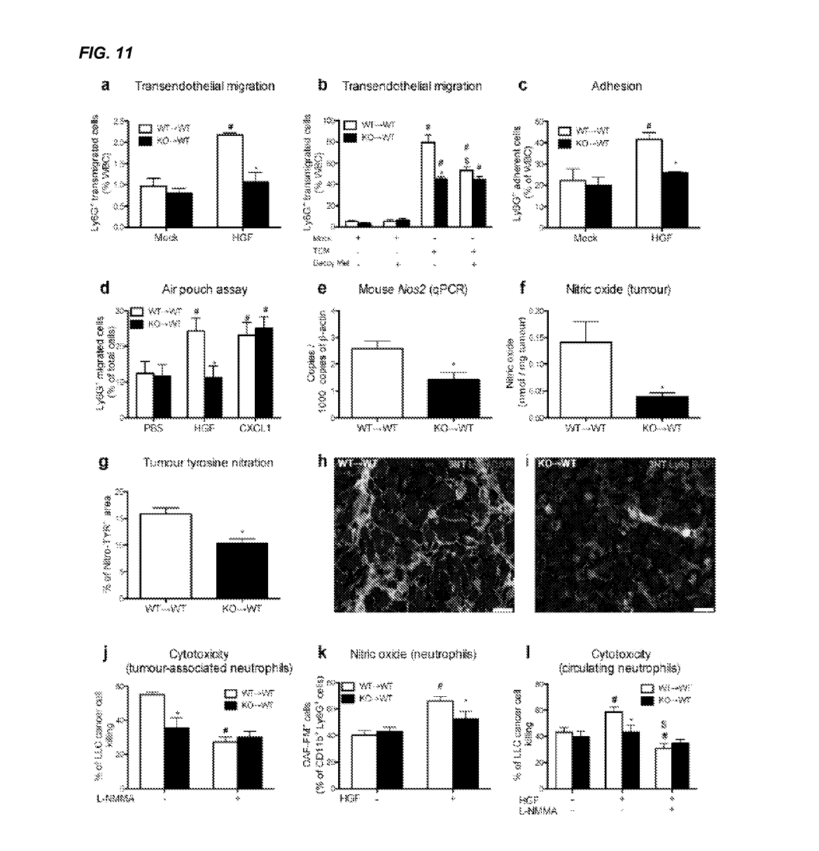

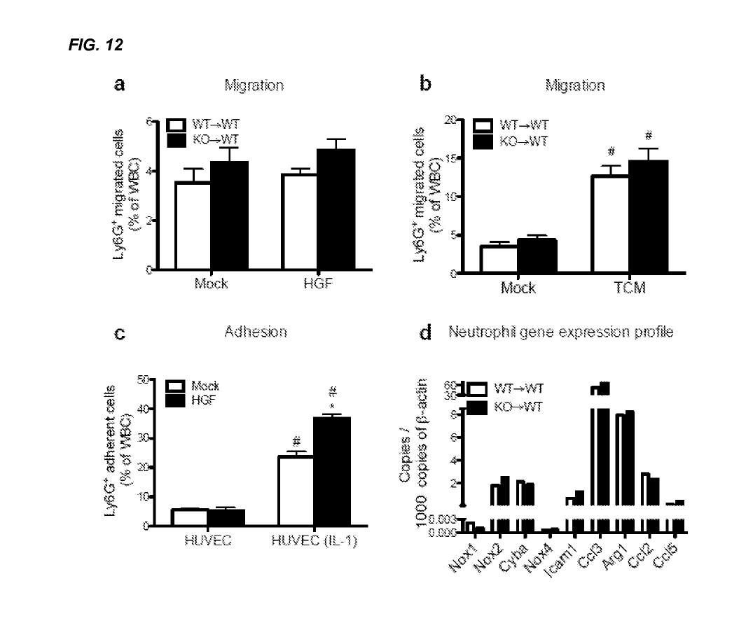

Surprisingly, tumor volume, tumor weight, lung metastasis, and total metastatic area of subcutaneous Lewis lung carcinomas (LLC) in KO→WT versus WT→WT mice were increased, respectively, 1.6, 1.4, 2.1, and 3.4-fold (FIGS. 1a-c and FIGS. 2a-c). The increased number of metastatic nodules in the lungs of KO→WT mice was not attributable to an increase in tumor growth only, since Met deficiency in the hematopoietic lineage raised the metastatic index (that is the number of metastases divided by tumor weight; FIG. 2d). Histological analyses revealed that, compared to WT→WT mice, KO→WT mice displayed reduced tumor apoptosis and necrosis, but increased proliferation (FIGS. 1d-l). Tumor vessel area, density, perfusion and oxygenation were comparable in both chimeric mice (FIGS. 2e-h). A similar induction in LLC tumor growth and metastasis were observed in Tie2;Met^{lox/lox} versus Tie2;Met^{wt/wt} mice (FIGS. 2i and 2j). This finding might have an important clinical outcome. Indeed, systemic delivery of Met inhibitors could foster a pro-tumor phenotype (or counteract an anti-tumor phenotype) in the hematopoietic lineage, inducing a possible mode of resistance to targeted therapy.

Of note, tumor growth, vessel area, density, perfusion and oxygenation in Tie2;Met^{lox/lox} mice reconstituted with WT BM cells (WT→KO), which results in EC-specific deletion of Met, were the same as those in WT→WT control mice (FIGS. 2k-o). This observation suggests that the role of MET in ECs, at least in this tumor model, is dispensable for tumor vessel formation and that the anti-angiogenic effect of HGF/MET inhibitors described so far, might be indirect and not EC autonomous ¹⁴.

To extend our finding to other tumor types, we monitored the growth of subcutaneous T241 fibrosarcomas, or orthotopic Panc02 pancreatic carcinomas in WT→WT and KO→WT mice, or of spontaneous metastatic mammary tumors in BM-transplanted MMTV-PyMT mice. Genetic deletion of Met in the hematopoietic system increased the growth of T241 fibrosarcomas and PyMT+ breast tumors (FIGS. 1m and 1n) while Panc02 pancreatic carcinomas grew similarly in WT→WT and KO→WT mice (FIG. 2p). The number of lung metastasis in MMTVPyMT mice, reconstituted with Met KO BM cells, was increased when compared to control MMTV-PyMT mice, reconstituted with WT BM cells (FIG. 1o).

### Example 2

### Met Deletion in the Hematopoietic Lineage Inhibits Neutrophil Recruitment to the Primary Tumor and Metastatic Niche

The numbers of circulating and tumor-infiltrating immune cells in WT→WT and KO→WT mice were characterized. Both counts and percentage of different circulating blood cell subsets were comparable in both chimeric mice (FIGS. 3a-c and Table 2).

TABLE 2
Blood count in WT→WT and KO→WT tumor-free or tumor-bearing
mice.

| | WT→WT | KO→WT |
|---|---|---|
| Tumor free | | |
| WBC (k/μl) | 10.03 ± 2.05 | 8.66 ± 0.93 |
| NEU (%) | 9.18 ± 2.1 | 10.3 ± 3.07 |
| LYM (%) | 85.2 ± 2.91 | 83.94 ± 3.46 |
| MON (%) | 1.41 ± 0.48 | 1.3 ± 0.4 |
| EOS (%) | 0.44 ± 0.17 | 0.25 ± 0.05 |
| BAS (%) | 3.77 ± 0.68 | 4.21 ± 0.18 |
| RBC (M/μl) | 8.21 ± 0.54 | 9.3 ± 0.21 |
| HCT (%) | 59.36 ± 3.52 | 70.16 ± 1.67 |
| MCHC (g/dl) | 18.63 ± 0.61 | 13.4 ± 0.16 |
| PLT (K/μl) | 589.26 ± 134.65 | 758.4 ± 50.63 |
| Tumor bearing | | |
| WBC (k/μl) | 7.97 ± 0.63 | 9.12 ± 1.22 |
| NEU (%) | 44.3 ± 0.37 | 53.71 ± 7.23 |
| LYM (%) | 27.29 ± 8.33 | 33.96 ± 2.52 |
| MON (%) | 1.79 ± 0.75 | 1.9 ± 0.64 |
| EOS (%) | 0.26 ± 0.03 | 0.45 ± 0.1 |
| BAS (%) | 1.94 ± 0.53 | 1.84 ± 0.57 |
| RBC (M/μl) | 5.5 ± 0.54 | 6.83 ± 0.46 |
| HCT (%) | 42.13 ± 2.93 | 49.43 ± 3.47 |
| MCHC (g/dl) | 18.27 ± 0.5 | 19.24 ± 0.05 |
| PLT (K/μl) | 566.17 ± 109.48 | 805.5 ± 88.19 |

When analyzing immunostained sections of endstage (i.e., 21 days) LLC tumors, infiltrating macrophages (FIG. 3d), natural killer (NK) cells (FIG. 3e), B lymphocytes (FIG. 3f), T helper (FIG. 3g), cytotoxic T lymphocytes (FIG. 3h) and dendritic cells (FIG. 3i) did not change but Ly6G+ neutrophil area was reduced by 73.4% in KO→WT mice (FIGS. 4a-c).

To assess if this difference in neutrophil infiltration upon Met deletion changes over time, we quantified Ly6G-positive areas 9, 13 or 19 days after LLC tumor implantation. In WT→WT mice, Ly6G+ cells decreased during tumor progression but Met KO neutrophils were anyhow fewer than their WT counterparts at all the time points tested (FIG. 4d). Neutrophil infiltration in T241 fibrosarcomas and PyMT+ breast tumors were 2.5 and 1.5-fold lower in KO→WT than WT→WT mice (FIGS. 4e and 4f). In Panc02 pancreatic carcinomas (where hematopoietic deletion of Met did not affect tumor growth), neutrophil infiltration was comparable in both WT→WT and KO→WT mice, but, in general, this tumor failed to induce a significant recruitment of neutrophils compared to the other tumor types (FIG. 3j). Consistent with a role of neutrophils in the inhibition of metastatic seeding ^{34,36}, Ly6G+ cells at the metastatic lungs of KO→WT mice were 33% lower than in WT→WT mice (FIGS. 4g-i).

These results disclose a possible tumor-inhibiting role for c-Met-positive granulocytes. As other inflammatory cells, granulocytes can have an anti-tumoral phenotype and directly kill tumor cells or release cytotoxic molecules like ROS or proteases or influence the recruitment of other immune cell types, but they can also be ejected by the cancer cells and favor tumor growth (Di Carlo et al., Blood 97, 339-45, 2001). It should be noted that modulation of pro-versus anti-tumoral phenotype of tumor-associated neutrophils by modulating TGF-b activity has recently been reported (Fridlender et al., Cancer Cell. 2009; 16(3):183-94). Without being bound to a particular mechanism, it is possible that c-Met is a marker for the anti-tumoral “N1” population, implying that upregulating c-Met activity in granulocytes or neutrophils would have a stronger anti-tumoral effect.

Innate and adaptive immunity may communicate and influence each other ³⁹. Thus, we used the myeloid-cell-specific deleter line, LysM:Cre (that is active in neutrophils and macrophages as well), to inactivate MET in cells of the innate immune system only. Genetic disruption of this pathway in myeloid cells accelerated the growth of subcutaneous LLC tumors (FIGS. 4j and 4k). This phenotype was associated with reduced neutrophil but unaltered macrophage infiltration to the tumor (FIG. 4l and FIG. 3k).

Myeloid cells can influence tumor growth by modulating lymphocyte activation ³⁹. To test this possibility, we transplanted WT and Met KO BM cells in athymic mice wherein the lack of thymus does not allow T cell maturation and partially affects B cell functions. Also in this case, MET deficiency in the hematopoietic lineage fostered LLC tumor growth (FIGS. 4m and 4n) and reduced neutrophil infiltration to the tumor (FIG. 4o). Overall, these results indicate that the anti-tumor activity of MET in hematopoietic cells (and more specifically in myeloid cells) does not need lymphocytes.

### Example 3

### Met Deletion in the Hematopoietic Lineage Inhibits Neutrophil Recruitment to the Inflammatory Site in Different Inflammation Models

Neutrophils are short-lived cells with a defined apoptotic program that is essential for the resolution of inflammation. Signs of neutrophil apoptosis are cell shrinkage, nuclear chromatin condensation, DNA fragmentation, and cell surface exposure of phosphatidylserine ⁴⁰. However, the reduction of intratumoral Ly6G+ cells in KO→WT mice was not due to a difference in apoptosis since TUNEL-positive or Annexin V-positive neutrophils did not change (FIGS. 5a and 5b).

To evaluate the effect of Met deletion on neutrophil recruitment from the bloodstream to the inflammatory site, we used a well-established model of acute skin inflammation, consisting in the application of the phorbol ester TPA or vehicle to each ear of WT→WT and KO→WT mice. After 24 hours, MET inactivation abated neutrophil infiltration into the inflamed skin by 62% (FIGS. 5c and 5d), whereas F4/80+ macrophages or CD3+ lymphocytes were equally recruited in both genotypes (FIGS. 5e and 5f). Similarly, induction of peritonitis in WT→WT mice (by intraperitoneal injection of the yeast cell wall derivative zymosan A) resulted in a massive recruitment of F4/80+ macrophages and Ly6G+ neutrophils after 4 hours. Peritoneal exudates harvested from KO→WT mice contained 5.2-fold less neutrophils than those from WT→WT mice, while macrophage count was not affected (FIG. 5g).

All these data indicate that MET is required for granulocyte (particularly neutrophil, since these make up the bulk of the granulocytes) recruitment to inflamed tissues or tumors, and that inhibition of the MET pathway decreases granulocyte transmigration.

### Example 4

### Inflammatory Stimuli and Tumor-Derived TNF-a Promote Met Expression in Neutrophils

To date, there is no evidence of Met expression in neutrophils. We, thus, thoroughly investigated by FACS and quantitative PCR analysis whether Met is expressed in circulating or tumor-infiltrating neutrophils. Both RNA and FACS analysis revealed that circulating Ly6G+ cells of healthy mice express low levels of MET. These levels were increased in circulating neutrophils of LLC tumor-bearing mice and even further in tumor-infiltrating neutrophils (FIGS. 6a-c). Interestingly, while RNA levels of c-Met were also scarce in lymphocytes and in circulating monocytes, and are also induced in tumor infiltrating macrophages, the observed expression increase is much stronger in granulocytes than that observed in macrophages or lymphocytes (FIG. 6l).

To test whether a similar upregulation of MET in neutrophils was preserved in humans, we isolated neutrophils from non-small cell lung tumors and healthy tissues from the same patient and we found that MET levels in tumor-infiltrating CD66b+CD11b+ neutrophils were 7.2-times higher than in neutrophils sorted from the healthy lung (FIG. 6d).

Based on these results, we wondered which factors were responsible for MET induction in neutrophils following tumor onset. Both MET RNA and protein were low in cultured naïve neutrophils, isolated from blood or BM of healthy mice. Co-culture with an activated inflamed-like endothelium (pre-stimulated with IL-1) as well as stimulation with conditioned medium from freshly harvested LLC tumors (TCM) or with medium harvested from cultured LLC cells (CCM), potently induced MET transcripts and protein in neutrophils (FIGS. 6e, f, j). The same effect was observed by stimulating human neutrophils with medium harvested from cultured A549 human lung cancer cells (FIGS. 6g and 6k). Co-culture of neutrophils with naïve endothelium or exposure to hypoxia-a condition known to induce Met in cancer cells 7- did not change Met expression in neutrophils (FIG. 6e and FIGS. 7a and 7b).

When seeking for the factors that can induce MET in naïve mouse neutrophils, we found that TNF-a or LPS (but not IL-1 or HGF) were able to upregulate MET at both RNA and protein levels (FIGS. 6h and 6j and not shown). The same effect of TNF-a or LPS was observed in human neutrophils as well (FIGS. 6i and 6k).

Then, we used one of the conditions where we observed Met upregulation in mouse neutrophils, namely neutrophil co-culture with activated ECs, and blocked TNF-a by different means. Silencing of EC-borne TNF-a (which is 250-fold increased upon stimulation with IL-1; FIG. 8a), pharmacological blockade of TNF-a with the TNF-a-trap Enbrel, or genetic knockout of TNFRI (but not of TNFRII), equally prevented Met induction in neutrophils upon co-culture with activated ECs (FIGS. 9a-c). Likewise, stimulation with TNF-a or LLC-derived TCM failed to upregulate Met in TNFRI KO neutrophils but not in TNFRII KO neutrophils (FIGS. 9d and 9e). In line, neutralization of TNF-a in LLC-TCM or in A549-CCM strongly abated Met induction in mouse or human neutrophils, respectively, (FIGS. 9f and 9g). Of note, TNF-an inhibition or genetic deletion of TNFRI in mouse neutrophils slightly downregulated the baseline levels of Met, suggesting that an autocrine loop of TNF-a partly sustains Met expression in resting conditions.

Overall, these data indicate that MET is strongly induced in neutrophils upon exposure to inflammatory stimuli such as tumor-derived TNF-a.

### Example 5

HGF-Mediated MET Activation in Neutrophils Triggers their Transendothelial Migration.

The endothelium represents a barrier to protect healthy tissues by non-specific reactions of the innate immune system ²⁶. Inflammatory cytokines upregulate adhesive molecules such as ICAM (intercellular adhesion molecule) and VCAM (vascular cell adhesion molecule) on the EC surface, which allow immune cells to transmigrate and reach their target tissue. To reach the tumor under the influence of several cytokines and chemokines, granulocytes begin rolling on the inner surface of the vessel wall before starting to adhere firmly; finally they transmigrate and migrate into the tissue by following the chemotactic gradients to the site of injury. By using granulocytes from WT and KO mice, we found that HGF increased the firm adhesion of granulocytes to an activated endothelium and this effect was mediated by c-Met: HGF stimulation of WT neutrophils promoted their chemotaxis through an inflamed-like endothelium; Met KO neutrophils completely lost this response to HGF (FIG. 11a). In general, HGF did not influence the migration of neutrophils through a naked porous membrane or a non-activated endothelium (FIG. 12a and not shown).

HGF is released in the extracellular milieu by tumor-associated stromal cells ⁴¹. Stimulation of WT neutrophils with TCM promoted transendothelial migration; administration of a soluble HGF-trap (decoy MET) consisting of the extracellular portion of MET ¹⁴ abated this effect, indicating that tumor-derived HGF is, at least in part, responsible for neutrophil migration through the endothelium. Notably, transendothelial migration of Met KO neutrophils in response to TCM was similar to that of TCM-stimulated WT neutrophils in presence of decoy MET. Decoy MET did not further impair the migration of Met KO neutrophils (FIG. 11b). TCM-induced neutrophil chemotaxis through naked filters (that were not coated with ECs) was comparable in both genotypes (FIG. 12b).

Transendothelial migration of neutrophils requires their tight adhesion to the inner surface of the vessel wall ²⁶. HGF increased the adhesion of WT neutrophils to an activated endothelium by 48%, but did not modify the behavior of KO cells (FIG. 11c). In general, neutrophil adhesion to nonactivated ECs was low and not affected by HGF (FIG. 12c).

The relevance of HGF-mediated MET activation during neutrophil transmigration through the vessel wall was tested using an air pouch model. Air pouches were raised on the dorsum of WT→WT and KO→WT mice. After 6 days, when an epithelial layer is formed, HGF or the well-known neutrophil chemoattractant CXCL1 were injected into the pouch. The exudates were then harvested and analyzed by FACS. HGF and CXCL1 were equally good in recruiting Ly6G+ cells. The recruitment of neutrophils towards HGF was completely abolished in KO→WT mice while the effect of CXCL1 did not change compared to that in WT→WT mice (FIG. 11d).

Altogether, HGF-mediated MET activation is required for neutrophil migration through an adhesive endothelium towards the inflammatory site.

### Example 6

### B2-Integrin is Part of the c-Met Granulocyte Adhesion Pathway

As activated integrins are known to be involved in the adhesion and diapedesis of granulocytes, the effect of blocking the β2-integrin on HGF-induced granulocyte adhesion was evaluated. Using the anti-CD18 antibody GAME-46 (BD Biosciences; Driessens et al., J Leukoc Biol. 1996; 60(6):758-765) that specifically inhibits β2-integrin, it could be shown that less granulocytes adhere compared to treatment with a control antibody (FIG. 10A). Moreover, stimulation with HGF increases the percentage of granulocytes bound to ICAM-1 in a soluble ICAM-1 binding assay (FIG. 10B); immunoprecipitation experiments show that there is more active Iβ2-integrin upon HGF stimulation (FIG. 10C).

### Example 7

### MET Promotes Nitric Oxide-Mediated Cytotoxicity in Neutrophils

Once migrated into the tumor, neutrophils can inhibit or favor tumor progression depending on their response to specific stimuli ²⁸. We hypothesized that the recruitment of neutrophils by tumor-released HGF might be associated to a switch of these neutrophils towards an anti-tumor/cytotoxic phenotype. For this reason, we measured the expression of anti-tumoral (N1) and pro-tumoral (N2) genes in tumor-infiltrating neutrophils freshly isolated from WT→WT and KO→WT mice. Among all, tumor-infiltrating neutrophils from KO→WT mice displayed 1.8-times lower expression of the N1-type gene inducible nitric oxide synthase (Nos2, also known as iNos) whereas other N1 genes ^{33,42,43} including Nox1, Nox2, the NOX3 subunit Cyba, Nox4, Icam1, and Cc13, or N2 genes 33, including Arg1, Cc12, and Cc15, did not change significantly (FIG. 11e and FIG. 12d). Consistently, tumors harvested from KO→WT mice showed reduced concentrations of nitric oxide (NO) in comparison to tumors from WT→WT mice (FIG. 11f).

As a sign of NO-mediated cytotoxicity, we measured the formation of 3-nitrotyrosine (3NT) in tumor sections and found that 3NT-positive tumor areas were 1.5-fold reduced in KO→WT versus WT→WT mice (FIGS. 11g-i). In vitro, intratumoral neutrophils freshly sorted from KO→WT mice had lower capacity to kill cancer cells, compared to intratumoral neutrophils sorted from WT→WT mice; pharmacological inhibition of iNOS by L-NMMA decreased the cytotoxicity of WT neutrophils to the levels of KO neutrophils (FIG. 11j).

We then provided proof that HGF is responsible for increased neutrophil cytotoxicity. To this end, WT and Met KO circulating neutrophils were incubated together with LLC cancer cells and stimulated with HGF or no factor. Basal NO production and cancer cell killing were comparable in both WT and Met KO neutrophils (FIGS. 11k and 11l). However, HGF treatment augmented NO release and cytotoxicity of WT, but not KO neutrophils. L-NMMA decreased HGF-induced cytotoxicity to the level of Met KO neutrophils (FIG. 11l).

Altogether, we show that HGF attracts neutrophils to the tumor where it triggers a cytotoxic response against cancer cells.

### Example 8

### Exploring c-Met Inhibition in Granulocyte-Mediated Inflammatory Disease

This is the first time that a distinct role for c-Met signaling in granulocyte adhesion and endothelial transmigration has been proposed. To our knowledge, c-Met inhibitors have up till now only been evaluated in cancer models. The results presented herein indicate that inhibition of c-Met signaling could have clear benefits in inflammatory disease where excess infiltration of granulocytes is a problem. To evaluate whether c-Met inhibition can have therapeutic benefits, we will test c-Met inhibitors in a mouse model of asthma and check both infiltration of granulocytes and clinical symptoms.

### DISCUSSION

Although the role of HGF/MET signaling in cancer cells is well established, little was known about MET expression and function in the immune system. This is important because immune cells restrain malignant cells to expand and disseminate but can also foster tumor development and metastasis ³⁹. In this study, we show for the first time that MET is induced, in both human and mouse granulocytes (of which neutrophils are by far the largest subset), during pathophysiological inflammation such as peritonitis, cancer, and cutaneous rash. MET is then required for granulocyte (neutrophil) migration through the vessel wall of inflamed tissues where neutrophils exert anti-microbial and anti-tumoral functions via NO and reactive oxygen species production, extracellular release of granule contents, and phagocytosis.

From an immunological point of view, the mechanism described in this study highlights a clever and fine control of non-specific immune reactions, which is necessary in order to prevent damage of healthy organs and, on the other hand, to confine this cytotoxic response to the site of inflammation only. Indeed, first, the endothelium must be activated by pro-inflammatory cytokines to allow neutrophil chemoattraction in general. Second, MET is induced and, thus, promotes neutrophil transendothelial migration. Third, the MET ligand HGF is expressed and proteolytically activated at the site of inflammation. Finally, migration of neutrophils towards the infection site, tumor nest, or metastatic niche favors neutrophil activation and HGF-mediated production of NO. Although other studies have reported MET expression in monocytes, macrophages, dendritic cells, and lymphocytes ²¹⁻²⁵, our data clearly suggest that, in vivo, HGF/MET pathway is indispensable for the recruitment of neutrophils, but not of other immune cells, during several inflammatory processes.

From a therapeutic point of view in the field of cancer, these findings imply that tumors that are not oncogene-addicted for MET might better escape the immune surveillance when a MET-targeted therapy is used. Thus, these patients might suffer, instead of benefit, from this pharmacologic approach. Data from clinical trials showed that an anti-MET antibody, blocking HGF binding to MET, decreased 3-fold the risk of death of non-small cell lung cancer patients with MET-high tumors (HR=037; 95% CI=0.20-0.71; p=0.002), but the overall survival of patients with low or no MET expression was reduced from 9.2 to 5.5 months (HR=3.02; 95% CI=1.13-8.11; p=0.021)⁴⁴. Our findings point towards patient stratification protocols, based on MET expression in cancer versus stromal cells in order to predict the population that has the highest chance to respond to MET-targeted therapies.

Most but not all the tumors tested in our study were infiltrated with abundant neutrophil exudate and this process was regulated by HGF/MET pathway. HGF is mainly secreted by mesenchymal cells, which release a precursor, pro-HGF, that requires activation by proteases, such as urokinase-type and tissue-type plasminogen activators (uPA and tPA, respectively)¹⁵. Different tissues and tumor types can be more or less rich in uPA and tPA, or express different amount of the plasminogen activator inhibitor (PAI), altogether affecting the level of pro-HGF cleavage. This might explain why some inflammatory conditions or tumor models are more sensitive to MET-dependent neutrophil recruitment than others. Alternatively, different tumor entities might have lower or higher ability to induce MET in neutrophils depending on the amount and type of proinflammatory cytokines released, such as TNF-a or others.

Previous literature has described anti-tumor effects (N1) and tumor-supportive roles (N2) of neutrophils ^{28-34,36,45}. In agreement with their biological functions, infiltration of neutrophils has been associated with either favorable or bad prognosis in different human tumors ²⁸. These opposing populations of neutrophils are not predefined subsets but they rather reflect the plasticity and versatility of these cells in response to microenvironmental signals. As the complexity and prevalence of specific signals fluctuate during cancer progression and depend on the tumor type, different progression stages or different tumor subsets can display N1-like or N2-like neutrophils. Neutrophil-associated pro-tumorigenic effects are mainly dependent on TGF-β signaling and its inhibition enables the N2 phenotype ³³. Here, we show that HGF/MET signaling is important for neutrophil recruitment to the tumor and NO-mediated cytotoxicity. As shown in the instant application, neutrophil recruitment to the metastatic niche is also greatly dependent on this pathway. Moreover, neutrophil infiltration inhibits metastasis ^{34,36}. It will be worthwhile to investigate if the anti-tumoral effect of neutrophils driven by MET activation can be overruled by excessive release of TGF-β by the tumor. In this case, the combination of anti-MET therapy and TGF-β inhibitors might result in a better therapeutic efficacy than each treatment alone.

Apart from cancer, neutrophil infiltration characterizes a diversity of autoimmune and/or inflammatory pathologies, including rheumatoid arthritis, asthma, chronic obstructive pulmonary disease, acute lung injury, and acute respiratory distress syndrome ⁴⁶⁻⁴⁸. In these disorders, neutrophil-derived reactive oxygen/nitrogen species as well as proteases are important effectors of tissue damage and disease progression. Our findings show that inhibition of MET results in a significant decrease of granulocyte/neutrophil recruitment to the inflammatory site (e.g., example 3). Thus, MET-targeted therapies could be used to treat or ameliorate the symptoms of pathologies characterized by high neutrophil or granulocyte infiltration, also given the fact that these drugs are not associated with overt toxicity or adverse reactions ^{3,14}. Conversely, current therapies such as TNF-a inhibitors have been reported to induce important side effects ^{49,50}. Notably, we show that MET is downstream TNF-a stimulation. Therefore, MET blockade is likely to prevent neutrophil recruitment and priming without affecting other cells wherein TNF-a plays instead a beneficial role. The results presented herein shed light on a novel role of MET in granulocytes, suggesting a possible mode of resistance to anti-MET treatments in cancer therapy and offering new opportunities for the improvement of these cancer therapies, as well as inflammatory diseases primarily mediated by granulocytes.

### REFERENCES

1. Trusolino, L., Bertotti, A. & Comoglio, P. M. MET signaling: principles and functions in development, organ regeneration and cancer. Nat Rev Mol Cell Biol 11, 834-848, (2010).
2. Birchmeier, C. & Gherardi, E. Developmental roles of HGF/SF and its receptor, the c-Met tyrosine kinase. Trends in cell biology 8, 404-410 (1998).
3. Gherardi, E., Birchmeier, W., Birchmeier, C. & Vande Woude, G. Targeting MET in cancer: rationale and progress. Nature reviews. Cancer 12, 89-103 (2012).
4. Tsuda, M. et al. TFE3 fusions activate MET signaling by transcriptional up-regulation, defining another class of tumors as candidates for therapeutic MET inhibition. Cancer research 67, 919-929 (2007).
5. Ivan, M., Bond, J. A., Prat, M., Comoglio, P. M. & Wynford-Thomas, D. Activated ras and ret oncogenes induce over-expression of c-met (hepatocyte growth factor receptor) in human thyroid epithelial cells. Oncogene 14, 2417-2423 (1997).
6. Toschi, L. & Cappuzzo, F. Clinical implications of MET gene copy number in lung cancer. Future oncology 6, 239-247 (2010).
7. Pennacchietti, S. et al. Hypoxia promotes invasive growth by transcriptional activation of the met protooncogene. Cancer Cell 3, 347-361 (2003).
8. Moghul, A. et al. Modulation of c-MET proto-oncogene (HGF receptor) mRNA abundance by cytokines and hormones: evidence for rapid decay of the 8 kb c-MET transcript. Oncogene 9, 2045-2052 (1994).
9. Kentsis, A. et al. Autocrine activation of the MET receptor tyrosine kinase in acute myeloid leukemia. Nat Med 18, 1118-1122 (2012).
10. Di Renzo, M. F. et al. Somatic mutations of the MET oncogene are selected during metastatic spread of human HNSC carcinomas. Oncogene 19, 1547-1555 (2000).
11. Comoglio, P. M., Giordano, S. & Trusolino, L. Drug development of MET inhibitors: targeting oncogene addiction and expedience. Nat Rev Drug Discov 7, 504-516 (2008).
12. Zillhardt, M., Christensen, J. G. & Lengyel, E. An orally available small-molecule inhibitor of c-Met, PF-2341066, reduces tumor burden and metastasis in a preclinical model of ovarian cancer metastasis. Neoplasia 12, 1-10 (2010).
13. Cecchi, F., Rabe, D. C. & Bottaro, D. P. Targeting the HGF/Met signaling pathway in cancer. European journal of cancer 46, 1260-1270 (2010).
14. Michieli, P. et al. Targeting the tumor and its microenvironment by a dual-function decoy Met receptor. Cancer Cell 6, 61-73 (2004).
15. Mazzone, M. et al. An uncleavable form of pro-scatter factor suppresses tumor growth and dissemination in mice. The Journal of clinical investigation 114, 1418-1432 (2004).
16. Gao, C. F. et al. Therapeutic potential of hepatocyte growth factor/scatter factor neutralizing antibodies: inhibition of tumor growth in both autocrine and paracrine hepatocyte growth factor/scatter factor:c-Met-driven models of leiomyosarcoma. Mol Cancer Ther 8, 2803-2810 (2009).
17. Petrelli, A. et al. Ab-induced ectodomain shedding mediates hepatocyte growth factor receptor down-regulation and hampers biological activity. Proc Natl Acad Sci USA 103, 5090-5095 (2006).
18. Ding, S., Merkulova-Rainon, T., Han, Z. C. & Tobelem, G. HGF receptor up-regulation contributes to the angiogenic phenotype of human endothelial cells and promotes angiogenesis in vitro. Blood 101, 4816-4822 (2003).
19. Bussolino, F. et al. Hepatocyte growth factor is a potent angiogenic factor which stimulates endothelial cell motility and growth. J Cell Biol 119, 629-641 (1992).
20. Liu, Y. et al. Hepatocyte growth factor and c-Met expression in pericytes: implications for atherosclerotic plaque development. J Pathol 212, 12-19 (2007).
21. Beilmann, M., Vande Woude, G. F., Dienes, H. P. & Schirmacher, P. Hepatocyte growth factor-stimulated invasiveness of monocytes. Blood 95, 3964-3969 (2000).
22. Galimi, F. et al. Hepatocyte growth factor is a regulator of monocyte-macrophage function. Journal of immunology 166, 1241-1247 (2001).
23. Chen, Q., DeFrances, M. C. & Zarnegar, R. Induction of met proto-oncogene (hepatocyte growth factor receptor) expression during human monocyte-macrophage differentiation. Cell Growth Differ 7, 821-832 (1996).
24. Baek, J. H., Birchmeier, C., Zenke, M. & Hieronymus, T. The HGF receptor/Met tyrosine kinase is a key regulator of dendritic cell migration in skin immunity. Journal of immunology 189, 1699-1707 (2012).
25. Adams, D. H. et al. Hepatocyte growth factor and macrophage inflammatory protein 1 beta: structurally distinct cytokines that induce rapid cytoskeletal changes and subset-preferential migration in T cells. Proc Natl Acad Sci USA 91, 7144-7148 (1994).
26. Borregaard, N. Neutrophils, from marrow to microbes. Immunity 33, 657-670 (2010).
27. Spicer, J. D. et al. Neutrophils promote liver metastasis via Mac-1-mediated interactions with circulating tumor cells. Cancer research 72, 3919-3927 (2012).
28. Fridlender, Z. G. & Albelda, S. M. Tumor-associated neutrophils: friend or foe? Carcinogenesis 33, 949-955, doi:10.1093/carcin/bgs123 (2012).
29. Tazzyman, S., Lewis, C. E. & Murdoch, C. Neutrophils: key mediators of tumor angiogenesis. Int J Exp Pathol 90, 222-231, doi:10.1111/j.1365-2613.2009.00641.x (2009).
30. Pekarek, L. A., Starr, B. A., Toledano, A. Y. & Schreiber, H. Inhibition of tumor growth by elimination of granulocytes. The Journal of experimental medicine 181, 435-440 (1995).
31. Nozawa, H., Chiu, C. & Hanahan, D. Infiltrating neutrophils mediate the initial angiogenic switch in a mouse model of multistage carcinogenesis. P Natl Acad Sci USA 103, 12493-12498, (2006).
32. Piccard, H., Muschel, R. J. & Opdenakker, G. On the dual roles and polarized phenotypes of neutrophils in tumor development and progression. Critical reviews in oncology/hematology 82, 296-309 (2012).
33. Fridlender, Z. G. et al. Polarization of tumor-associated neutrophil phenotype by TGF-beta: “N1” versus “N2” TAN. Cancer Cell 16, 183-194 (2009).
34. Granot, Z. et al. Tumor entrained neutrophils inhibit seeding in the premetastatic lung. Cancer Cell 20, 300-314 (2011).
35. di Carlo, E. et al. Neutrophils in anti-cancer immunological strategies: old players in new games. J Hematother Stem Cell Res 10, 739-748 (2001).
36. Lopez-Lago, M. A. et al. Neutrophil chemokines secreted by tumor cells mount a lung antimetastatic response during renal cell carcinoma progression. Oncogene 32, 1752-1760 (2013).
37. Huh, C. G. et al. Hepatocyte growth factor/c-met signaling pathway is required for efficient liver regeneration and repair. P Natl Acad Sci USA 101, 4477-4482 (2004).
38. Takeda, Y. et al. Macrophage skewing by Phd2 haplodeficiency prevents ischaemia by inducing arteriogenesis. Nature 479, 122-126 (2011).
39. DeNardo, D. G., Andreu, P. & Coussens, L. M. Interactions between lymphocytes and myeloid cells regulate pro-versus anti-tumor immunity. Cancer metastasis reviews 29, 309-316 (2010).
40. Zhu, D. et al. Deactivation of phosphatidylinositol 3,4,5-trisphosphate/Akt signaling mediates neutrophil spontaneous death. P Natl Acad Sci USA 103, 14836-14841 (2006).
41. Straussman, R. et al. Tumor micro-environment elicits innate resistance to RAF inhibitors through HGF secretion. Nature 487, 500-504 (2012).
42. Di Carlo, E. et al. The combined action of IL-15 and IL-12 gene transfer can induce tumor cell rejection without T and NK cell involvement. Journal of immunology 165, 3111-3118 (2000).
43. Bedard, K. & Krause, K. H. The NOX family of ROS-generating NADPH oxidases: physiology and pathophysiology. Physiol Rev 87, 245-313 (2007).
44. Spigel, D. R. et al. Final efficacy results from OAM4558g, a randomized phase II study evaluating MetMAb or placebo in combination with erlotinib in advanced NSCLC. J Clin Oncol 29, 7505 (2011).
45. Di Carlo, E. et al. The intriguing role of polymorphonuclear neutrophils in anti-tumor reactions. Blood 97, 339-345 (2001).
46. Gernez, Y., Tirouvanziam, R. & Chanez, P. Neutrophils in chronic inflammatory airway diseases: can we target them and how? The European respiratory journal 35, 467-469 (2010).
47. Grommes, J. & Soehnlein, 0. Contribution of neutrophils to acute lung injury. Mol Med 17, 293-307, (2011).
48. Wright, H. L., Moots, R. J., Bucknall, R. C. & Edwards, S. W. Neutrophil function in inflammation and inflammatory diseases. Rheumatology 49, 1618-1631 (2010).
49. Hastings, R. et al. Neutropenia in patients receiving anti-tumor necrosis factor therapy. Arthritis Care Res (Hoboken) 62, 764-769 (2010).
50. Bongartz, T. et al. Anti-TNF antibody therapy in rheumatoid arthritis and the risk of serious infections and malignancies: systematic review and meta-analysis of rare harmful effects in randomized controlled trials. JAMA: the journal of the American Medical Association 295, 2275-2285 (2006).
51. Cotter, M. J., Norman, K. E., Hellewell, P. G. & Ridger, V. C. A novel method for isolation of neutrophils from murine blood using negative immunomagnetic separation. The American journal of pathology 159, 473-481 (2001).
52. Haslett, C., Guthrie, L. A., Kopaniak, M. M., Johnston, R. B., Jr. & Henson, P. M. Modulation of multiple neutrophil functions by preparative methods or trace concentrations of bacterial lipopolysaccharide. The American journal of pathology 119, 101-110 (1985).

1. A method of modulating trans-endothelial migration and recruitment of granulocytes, the method comprising:
modulating the c-Met pathway in granulocytes.

2. The method of claim 1, wherein granulocyte trans-endothelial transmigration is enhanced by enhancing the c-Met pathway.

3. The method of claim 2, wherein enhancing of the c-Met pathway comprises increasing β2-integrin expression and/or activation.

4. The method of claim 3, comprising increasing β2-integrin activation by an antibody.

5. The method of claim 3, wherein increasing β2-integrin activation is done in presence of a c-Met inhibitor.

6. The method of claim 5, wherein the c-Met inhibitor is an antibody.

7. The method of claim 1, wherein the granulocytes are neutrophils.

8. A method of treating a subject suffering from cancer, the method comprising:
enhancing trans-endothelial migration and recruitment of granulocytes in the subject by enhancing the c-Met pathway in granulocytes via increasing β2-integrin activation utilizing an antibody in the presence of a c-Met inhibitor to enhance granulocyte trans-endothelial transmigration in the subject.

9. The method of claim 8, wherein the cancer is c-Met inhibitor resistant cancer.

10. The method of claim 1, wherein granulocyte transmigration is decreased by inhibiting the c-Met pathway.

11. The method of claim 10, wherein inhibition of c-Met comprises utilizing an antibody.

12. A method of treating a subject with a granulocyte-mediated inflammatory disease, the method comprising:
decreasing trans-endothelial migration and recruitment of the subject's granulocytes by inhibiting the c-Met pathway in the granulocytes.

13. A medicament comprising:
a c-Met inhibitor, and
a granulocyte transmigration stimulating factor.

14. A method of treating a subject suffering from cancer, the method comprising:
administering to the subject a combination comprising:
a c-Met inhibitor, and
a granulocyte transmigration stimulating factor.

15. The combination of claim 13, wherein the c-Met inhibitor is an antibody.

16. The combination of claim 13, wherein the granulocyte transmigration stimulating factor is a β2-integrin activator comprising an antibody.

17. A method of treating a subject suffering from a granulocyte-mediated inflammatory disease, the method comprising:
administering to the subject a c-Met inhibitor to treat the granulocyte-mediated inflammatory disease.

18. The method according to claim 6, wherein the c-Met inhibitor is onartuzumab (METMab) antibody.

19. The method according to claim 10, wherein granulocyte transmigration is decreased by inhibiting the c-Met pathway by inhibiting c-Met.

20. The medicament of claim 13, wherein the granulocyte transmigration stimulating factor is a β2-integrin activator.

21. The method according to claim 14, wherein the granulocyte transmigration stimulating factor is a β2-integrin activator.

22. The combination of claim 15, wherein the c-Met inhibitor is onartuzumab (METMab) antibody.

23. The method according to claim 17, wherein the c-Met inhibitor is a c-Met inhibitory antibody.
